(19) United States
(12) Patent Application Publication
(10) Pub. No.: US 2012/0165604 A1
(43) Pub. Date: Jun. 28, 2012

(54) ENDOLUMINAL FOLD CREATION

(75) Inventors: Michael J. Stokes, Cincinnati, OH (US); Mark S. Zeiner, Mason, OH (US); Jason L. Harris, Mason, OH (US); Gary L. Long, Cincinnati, OH (US)

(73) Assignee: Ethicon Endo-Surgery, Inc., Cincinnati, OH (US)

(21) Appl. No.: 12/975,685

(22) Filed: Dec. 22, 2010

(51) Int. Cl. A61B 1/00; A61B 18/18

(52) U.S. Cl. 600/104; 606/41

(57) ABSTRACT

Devices and methods are provided for forming one or more plications in the walls of a body cavity. In particular, endoscopic devices and methods are provided for forming and/or securing endoluminal tissue folds to reduce the volume of the gastric cavity. An end effector that includes a tissue receiving cavity can be delivered to a desired surgical site to allow a tissue fold to be formed within the tissue receiving cavity. A fastener can be used to secure the adjacent layers of tissue that form the tissue fold. The tissue folds can be effective to limit the stomach's capacity and create a feeling of satiety.

### FIELD OF THE INVENTION

The present invention relates generally to devices and methods for performing surgical procedures, and more particularly to endoscopic devices and methods for forming an endoluminal fold to reduce the volume of the gastric cavity.

### BACKGROUND OF THE INVENTION

Obesity is a serious medical condition that affects more than 30% of the U.S. population and contributes significantly to morbidity and mortality. Complications associated with obesity include hypertension, diabetes, coronary artery disease, stroke, congestive heart failure, multiple orthopedic problems, pulmonary insufficiency, and markedly decreased life expectancy. Additionally, obesity often affects an individual's quality of life. Accordingly, the monetary, physical, and psychological costs associated with obesity can be substantial. In fact, it is estimated that costs related to obesity exceed more than 100 billion dollars annually.

A variety of bariatric surgical procedures have been developed to treat obesity, the most common of which is the Roux-en-Y gastric bypass (RYGB). In a RYGB procedure, a small stomach pouch is separated from the remainder of the gastric cavity and attached to a resectioned portion of the small intestine. However, because this complex procedure requires a great deal of operative time, as well as extended and often painful post-operative recovery, the RYGB procedure is generally only utilized to treat people with morbid obesity.

In view of the highly invasive nature of the RYGB procedure, other less invasive bariatric procedures have been developed such as the Fobi pouch, bilio-pancreatic diversion, gastroplasty (“stomach stapling”), and gastric banding. In addition, implantable devices are known which limit the passage of food through the stomach. Gastric banding procedures, for example, involve the placement of a small band around the stomach near the junction of the stomach and the esophagus to restrict the passage from one part of the digestive tract to another, thereby affecting a patient's feeling of satiety.

While the above-described bariatric procedures are commonly used for the treatment of morbid obesity (i.e., greater than 100 pounds over one's ideal body weight), the risks of these procedures often outweigh the potential benefits for the growing segment of the population that is considered overweight. The additional weight carried around by these persons can still result in significant health complications but does not justify more invasive treatment options. However, because conservative treatment with diet and exercise alone may be ineffective for reducing excess body weight, treatment options should involve a less invasive, lower cost solution for weight loss.

It is known to create cavity wall plications though endoscopic procedures. However plication depth has traditionally suffered in transesophageal procedures due to the size restrictions of the endoscopic lumen. Endoluminal approaches are restricted by the rigid length and diameter that can be reliably and safely passed transorally into the stomach. Furthermore, access and visibility within the gastric and peritoneal cavities is limited in a purely endoscopic procedure as the extent of the reduction increases.

With the foregoing in mind, it is desirable to have a surgical weight loss procedure that is inexpensive, with few potential complications, and that provides patients with a weight loss benefit while buying time for the lifestyle changes necessary to maintain the weight loss. Further, it is desirable that the procedure be performed endoluminally in order to be less invasive to the patient, allowing for a quick recovery and less scaring. Additionally, it is desirable to have an apparatus for forming deep serosa to serosa tissue folds within the gastric lumen while limiting the potential risk to adjacent organs and tissue. Furthermore, it is desirable to have an apparatus for forming tissue plications in which the rigid length and diameter of the apparatus is minimized for transoral passage, yet can form tissue folds that are deeper than the initial rigid length of the apparatus. Yet further still, it is desirable to have apparatus for forming tissue plications within a gastric lumen which is of reduced complexity and which is combinable with a tissue anchoring device to facilitate secure tissue plications through a minimal number of transoral intubations.

### SUMMARY OF THE INVENTION

The present invention provides surgical devices and methods for forming one or more plications (i.e., tissue folds) in the wall of the gastric cavity. The plication(s) can be effective to reduce the volume of the gastric cavity, thereby limiting the stomach's capacity and creating a feeling of satiety. The plication(s) can also alter gastric motility to reduce the efficiency by which the stomach contributes to the digestion of food. The plication(s) can be comprised to create serosa-to-serosa apposition within at least a portion of the infolded region.

In one embodiment, an endoscopic instrument is provided having an elongate, flexible shaft having proximal and distal ends, an end effector disposed at the distal end of the elongate shaft, a tissue manipulator, and a fastener for securing adjacent layers of tissue. The end effector includes a tissue receiving cavity that has a first length in a delivery configuration, and a second length in a treatment configuration that is greater than the first length. The tissue manipulator is associated with the end effector and is configured to engage tissue to enable the tissue to be positioned within the tissue receiving cavity in the treatment configuration to create a tissue fold. The fastener is configured to secure adjacent layers of tissue that form the tissue fold disposed within the tissue receiving cavity. The tissue fold can have a depth greater than the first, delivery length of the tissue receiving cavity.

The end effector can include first and second jaws that define the tissue receiving cavity. In one embodiment, the jaws can be extendible such that the jaws can be moved between the delivery configuration to the treatment configuration. In one embodiment, the jaws can be disposed within a movable sheath in the delivery configuration, and the sheath can be proximally retracted in the treatment configuration. The first length of the tissue receiving cavity can be substantially zero.

The tissue manipulator is configured to engage tissue and can be axially extendible and retractable. In one embodiment, the tissue manipulator includes a distal tip that is configured to removably couple to tissue. The distal tip can be, for example, one or more of a corkscrew, vacuum port, and clamp. In one aspect, the endoscopic instrument can also include a stabilization element that is configured to constrain lateral movement of the tissue manipulator. For example, the stabilization element can be a sheath disposed around the tissue manipulator or a housing coupled to the tissue manipulator and slidably coupled to the end effector.

In another aspect, an endoscopic instrument is provided having an elongate, flexible shaft having proximal and distal ends, an end effector having first and second arms disposed at the distal end of the shaft, a tissue manipulator, and a fastener configured to secure adjacent layers of tissue. The end effector can be movable between a delivery configuration and a treatment configuration and the first and second arms can define a tissue receiving cavity in the treatment configuration. The tissue manipulator can be configured to engage tissue to enable the tissue to be positioned within the tissue receiving cavity in the treatment configuration to create a tissue fold. The fastener can be configured to secure adjacent layers of tissue that form the tissue fold disposed within the tissue receiving cavity. The tissue fold can have a depth greater than the maximum rigid length of the end effector in the delivery configuration. In one aspect, the end effector can have a maximum rigid length in the treatment configuration that is greater than the maximum rigid length in the delivery configuration.

The arms of the end effector can have a variety of configurations. In one embodiment, at least one of the arms of the end effector can be a segmented arm formed of a plurality of connected link segments. The link segments can be flexibly coupled relative to one another in the delivery configuration to form a flexible segmented arm. In another aspect, the link segments can be rigidly coupled relative to one another in the treatment configuration to form a rigid segmented arm. In another aspect, the endoscopic instrument can include an actuator coupled to the segmented arm and configured to move the segmented arm between a flexible configuration and a rigid configuration.

In other aspects, a method for forming an endoluminal fold of tissue within a body cavity is provided. The method can include inserting an instrument having a flexible shaft and an end effector into a body cavity through a natural opening in a body. The end effector can have first and second jaws that are effective to define a tissue receiving cavity having a first length in a delivery configuration. The end effector can be manipulated to cause the tissue receiving cavity to have a second length greater than the first length. The method can also include manipulating tissue to create a tissue fold that is positioned within the tissue receiving cavity and fastening the tissue fold to secure adjacent layers of tissue that form the tissue fold. The length of the tissue fold can be substantially the same as the second length and greater than the first length.

The end effector can be manipulated to cause the tissue receiving cavity to have a second length greater than the first length in various ways. In one aspect, the end effector can be manipulated by manipulating the jaws. In one embodiment, the jaws can be distally extended. For example, extension members coupled to the jaws can be distally extended. In another aspect, the end effector can be manipulated by retracting a sheath disposed around the jaws in the delivery configuration, such that the jaws extend distally from the sheath.

In one aspect, tissue can be manipulated to create a tissue fold that is positioned within the tissue receiving cavity. In one embodiment, manipulating the tissue comprises removably engaging tissue with a tissue manipulator associated with the end effector. The tissue manipulator can be retracted to pull the tissue within the tissue receiving cavity. Further, the jaws of the end effector can be manipulated before or after the tissue manipulator is retracted.

In one aspect, the depth of the tissue fold can be substantially the same as the second length of the tissue receiving cavity and greater than the first length of the tissue receiving cavity in the delivery configuration. In one aspect, the depth of the tissue fold can be greater than the maximum length of the end effector in the delivery configuration.

In one aspect, the tissue fold can be fastened by securing adjacent layers of tissue that form the tissue fold. For example, a tissue fastener can be inserted through the adjacent layers of tissue that form the tissue fold. In another aspect, energy can be applied to the tissue fold to bond the adjacent layers.

In other aspects, a method for forming an endoluminal fold of tissue within a body cavity is provided. The method can include inserting an instrument having a flexible shaft and an end effector into a body cavity through a natural opening in a body. The end effector can include a tissue receiving cavity and at least one electrode configured to deliver energy to tissue disposed in the tissue receiving cavity. The end effector can be positioned adjacent tissue to be treated and the tissue can be manipulated to create a tissue fold that is disposed within the tissue receiving cavity. The method can also include delivering energy to the tissue fold disposed within the tissue receiving cavity through the at least one electrode such that adjacent layers of the tissue that form the tissue fold are secured. In one embodiment, the end effector can be movable between a delivery configuration and a treatment configuration, wherein the tissue fold has a depth greater than a length of the tissue receiving cavity in the delivery configuration.

Various types of energy can be employed to secure the layers of tissue. For example, RF energy or ultrasonic energy can be applied to the tissue fold to bond the tissue layers.

In one aspect, delivering energy to the tissue fold can include contacting opposed serosal layers of the tissue fold and applying energy thereto to bond the opposed layers. The at least one electrode for delivering the energy can have a variety of configurations. In one embodiment, the at least one electrode can be a needle that is movably disposed within one of the jaws of the end effector. The needle can be activated after the needle is inserted through at least a portion of the tissue that forms the tissue fold. The needle can be inserted substantially along a central axis of the tissue fold, or substantially transverse to the tissue fold. In one embodiment, the needle can be inserted through both of the adjacent layers of the tissue that form the tissue fold.

In other aspects, an endoscopic instrument is provided that can include an elongate, flexible shaft having proximal and distal ends, an end effector having a tissue receiving cavity and at least one electrode associated therewith effective to deliver energy to tissue disposed in the tissue receiving cavity, and a tissue manipulator configured to engage tissue to enable tissue to be disposed within the tissue receiving cavity to create a tissue fold. The endoscopic instrument can also include an actuator effective to cause energy to be delivered through the at least one electrode to the tissue fold such that adjacent layers of the tissue that form the tissue fold can be secured.

The end effector can be disposed at the distal end of the shaft and can have various configurations. For example, the end effector can be movable from a delivery configuration to a treatment configuration, wherein the tissue fold has a depth greater than a length of the tissue receiving cavity in the delivery configuration. In one aspect, the end effector can include first and second jaws. The electrode can be movable from a first position substantially within one of the jaws, to a second position at least partially within the tissue receiving cavity. The electrode is configured to penetrate tissue within the tissue receiving cavity as the electrode moves from the first configuration to the second configuration. In one aspect, the electrode can be configured to penetrate tissue substantially transverse to the tissue fold, or alternatively, the electrode can be configured to penetrate tissue substantially along a central axis of the tissue fold. In one aspect, the electrode can be configured to penetrate both of the adjacent layers of the tissue that form the tissue fold.

The electrodes can also be configured to deliver various types of energy to secure the layers of tissue. For example, the electrodes can be configured to deliver radiofrequency (RF) or ultrasonic energy to bond the tissue layers.

### BRIEF DESCRIPTION OF THE DRAWINGS

FIG. 1 illustrates one exemplary embodiment of a surgical device effective to create an endoluminal tissue fold, the device being disposed through an endoscope inserted into the upper gastrointestinal tract of a patient;

FIG. 2 is a perspective view of the distal portion of the surgical device of FIG. 1 disposed within the gastric cavity;

FIG. 3 is a perspective view of the distal end of the fastener of FIG. 2;

FIG. 4A is a partial cross-sectional view of the end effector of the surgical device of FIG. 1;

FIG. 4B is a partial cross-sectional view of the end effector of the surgical device of FIG. 1 with a tissue fold disposed therein;

FIG. 5 is a perspective view of the end effector of the surgical device of FIG. 1, showing a tissue anchor being deployed into a tissue fold disposed within the end effector;

FIG. 6 is a sectional, perspective view of a gastric cavity showing a tissue fold formed in the anterior wall of the cavity;

FIG. 7A is a side view of one embodiment of an end effector for use with the surgical device of FIG. 1, the end effector having opposed arms and a tissue manipulator effective to engage tissue;

FIG. 7B is a side view of the end effector of FIG. 7A showing the tissue manipulator being retracted to initiate a tissue fold;

FIG. 7C is a side view of the end effector of FIG. 7A showing the tissue manipulator fully retracted such that a tissue fold is disposed between the opposed arms;

FIG. 8A is a side view of another embodiment of an end effector for use with the surgical device of FIG. 1, the end effector having opposed arms and a tissue manipulator effective to engage tissue;

FIG. 8B is a side view of the end effector of FIG. 8A showing the tissue manipulator fully retracted such that a tissue fold is disposed between the opposed arms;

FIG. 8C is a side view of the end effector of FIG. 8A showing the opposed arms of the tissue manipulator being extended to lengthen the tissue fold;

FIG. 9A is a side view of another embodiment of an end effector for use with the surgical device of FIG. 1, the end effector having opposed arms and a tissue manipulator effective to engage tissue;

FIG. 9B is a side view of the end effector of FIG. 9A showing the opposed arms being extended to initiate a tissue fold;

FIG. 9C is a side view of the end effector of FIG. 9A showing the opposed arms fully extended with a tissue fold disposed therebetween;

FIG. 9D is a side view of the end effector of FIG. 9A showing the opposed arms fully extended and the tissue manipulator fully retracted;

FIG. 10 is a side view of another embodiment of an end effector for use with the surgical device of FIG. 1;

FIG. 11A is a side view of another embodiment of an end effector for use with the surgical device of FIG. 1, the end effector having a sheath disposed therearound;

FIG. 11B is a side view of the end effector of FIG. 11A showing the sheath and tissue manipulator being retracted to initiate a tissue fold;

FIG. 11C is a side view of the end effector of FIG. 11A showing the sheath and tissue manipulator fully retracted such that a tissue fold is disposed between the opposed arms;

FIG. 12A is a side view of another embodiment of an end effector for use with the surgical device of FIG. 1, the end effector having a tissue manipulator effective to engage tissue;

FIG. 12B is a side view of the end effector of FIG. 12A showing one of the opposed arms in a treatment configuration such that the tissue fold is disposed between the opposed arms;

FIG. 13A is a side view of another embodiment of an end effector for use with the surgical device of FIG. 1;

FIG. 13B is a perspective view of the end effector of FIG. 13A;

FIG. 14A is a perspective view of another embodiment of an end effector for use with the surgical device of FIG. 1, the end effector having opposed arms in a low-profile configuration;

FIG. 14B is a perspective view of the end effector of FIG. 14A showing the opposed arms in an open, delivery configuration;

FIG. 14C is a perspective view of the end effector of FIG. 14A showing the opposed arms in a closed configuration;

FIG. 14D is a partial cross-sectional side view of the end effector of FIG. 14A showing the opposed arms in a closed configuration with a tissue fold disposed therebetween;

FIG. 15 is a perspective view of another embodiment of an end effector for use with the surgical device of FIG. 1, the end effector having a tissue manipulator effective to engage tissue;

FIG. 16A is a cross-sectional view of the end effector of FIG. 15 showing the tissue manipulator in a retracted position such that a tissue fold is disposed within the end effector;

FIG. 16B is cross-sectional view of the end effector of FIG. 15 showing a fastener being applied to the tissue fold;

FIG. 16C is a cross-sectional view of the end effector of FIG. 15 showing a fastener disposed around the tissue fold to secure the tissue fold;

FIG. 17 is a side view of another embodiment of an end effector for use with the surgical device of FIG. 1, the end effector having an electrode for delivering energy to a tissue fold disposed within the end effector;

FIG. 18 is a side view of another embodiment of an end effector for use with the surgical device of FIG. 1, the end effector having an electrode for delivering energy to a tissue fold disposed within the end effector;

FIG. 19A is a perspective view of another embodiment of an end effector for use with the surgical device of FIG. 1, the end effector having an electrode for delivering energy to a tissue fold disposed therein; and

FIG. 19B is a partial cross-sectional side view of the end effector of FIG. 19A with a tissue fold disposed within the tissue receiving cavity of the end effector.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment can be combined with the features of other embodiments. Such modifications and variations are intended to be included within the scope of the present invention.

Various exemplary methods and devices are provided for creating a tissue fold to reduce the volume of the gastric cavity. The devices can be inserted transorally into the gastric cavity to involute and secure one or more sections of the cavity wall to form a serosa-to-serosa tissue fold. The devices described herein for forming an endoluminal tissue fold can have a number of different configurations but are generally designed to minimize the rigid length and diameter of the device to allow for transoral passage while maximizing the obtainable fold depth. Although in certain exemplary embodiments, the surgical device generally includes or is associated with a flexible shaft for insertion into the gastric cavity of a patient and an end effector for creating a serosa-to-serosa tissue fold, and a fastener for securing adjacent layers of tissue that form the serosa-to-serosa tissue fold, a person skilled in the art will appreciate that the concepts described herein can be applied to other surgical, therapeutic, or diagnostic devices in which it is desirable to form and secure tissue plications.

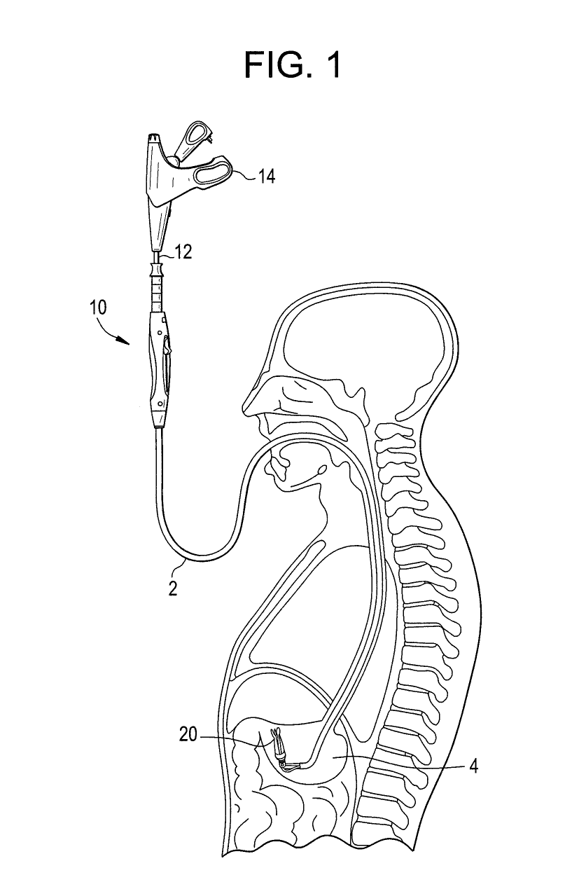

FIGS. 1-6 depict one exemplary embodiment of a surgical instrument for creating an endoluminal fold. As illustrated in FIG. 1, the surgical instrument 10 can be disposed within a natural body lumen to deliver the end effector 20 to a surgical site within the gastric cavity. As shown, the surgical instrument 10 can be disposed within an overtube or endoscope 2 for transesophageal access to a surgical site within the gastric cavity 4 of a patient. Although the illustrated surgical instrument 10 is shown delivered to the surgical site through the mouth and esophagus, it will be appreciated by a person skilled in the art that the surgical instrument 10 can be delivered to an internal surgical site through any opening within a patient's body, whether a natural orifice (e.g., orally, anally, or vaginally) or a surgical opening (e.g., through a trocar in a percutaneous incision, percutaneously, laparascopically, through a hybrid laparascopic-endoscopic approach, or an open surgical procedure). The surgical instrument 10 can also be delivered to a surgical site through the orifice or opening directly, without the use of an auxiliary endoscope, trocar, or other access port.

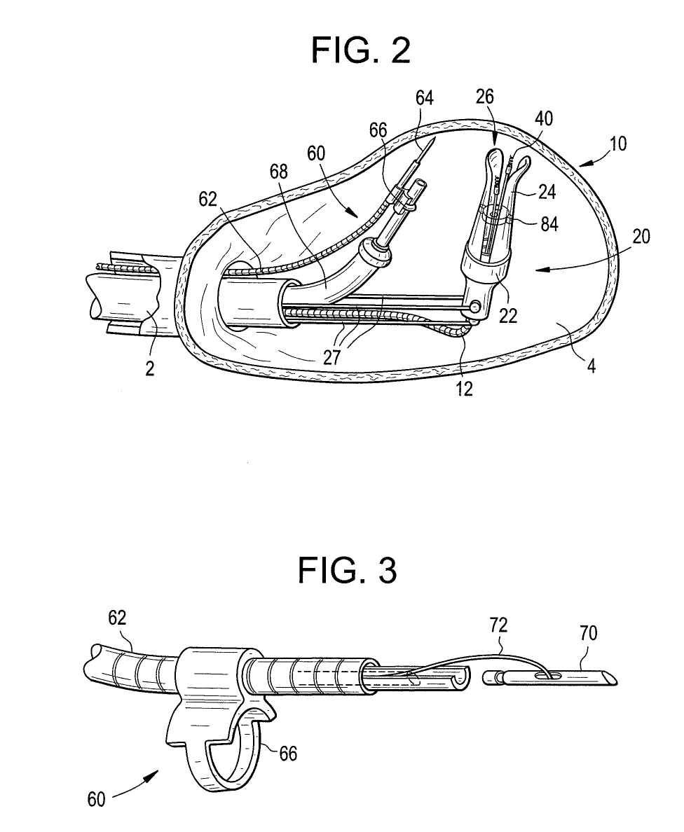

As shown in more detail in FIG. 2, the surgical instrument 10 generally includes an elongate shaft 12, an end effector 20 disposed at the distal end of the elongate shaft 12, and a tissue manipulator 40. The end effector 20 can define a tissue receiving cavity 26 in which a tissue fold can be formed. The tissue manipulator 40 can be configured to engage tissue to enable tissue to be positioned within the tissue receiving cavity 26 to form the tissue fold. The surgical instrument 10 can also include a fastener 60 associated therewith that is configured to secure adjacent layers of the tissue fold. As will be described in detail below, the fastener 60 can be integral with or coupled to the surgical instrument 10 itself or can be delivered and/or manipulated independent of the end effector 20.

Again referring to FIG. 1, the surgical instrument 10 can optionally include a handle assembly 14 operatively coupled to the end effector 20 and disposed external to the patient's body for facilitating control of the surgical device 10 and/or operation of the end effector 20. A person skilled in the skill in the art will appreciate that any of the various handle assemblies known in the art can be used including, for example, scissor-grip, pistol-grip, spool style handles, syringe style handles, and various other handle configurations modified in accord with the teachings herein. Alternatively, the user can operate the surgical device 10 manually, i.e., without the use of a handle assembly. By way of non-limiting example, the user can operate the end effector 20 by actuating controls (e.g., control wires, cables, etc.) that are configured to manipulate the end effector 20 directly without the need for a handle assembly.

The elongate shaft 12 is generally configured to extend from external to the patient's body to the surgical site and can have a variety of configurations. Generally, as shown in FIG. 1, the end effector 20 can be disposed on the distal end of the elongate shaft 12 and the proximal end of the elongate shaft 12 can extend outside the patient's body. The elongate shaft 12 can also define a lumen through which a variety of actuating mechanisms (e.g., cables, control wires, etc.) can extend for operating the end effector 20. A person skilled in the art will appreciate that any of the various shafts for endoscopic, laparoscopic, percutaneous, and other minimally invasive surgical devices known in the art can be modified in accord with the teachings herein for use with the present invention.

In an exemplary embodiment, at least a portion of the elongate shaft 12 is flexible or semi-flexible to allow the shaft 12 to be inserted into a patient translumenally, e.g., through a natural orifice, an endoscope, or a surgical incision. While various materials and techniques can be used to form the shaft, the elongate shaft 12 can be formed, for example, from a friction reducing flexible outer sheath having a flat coil wire extending therethrough. The flexibility of the shaft 12 can also vary along its length and the shaft 12 can be formed from one or more components that are mated together.

As will be described in detail below, the end effector for use with the surgical device of the present invention can have a variety of configurations, but generally includes a tissue receiving cavity in which a tissue fold can be formed. As shown in FIG. 2, the end effector 20 can include a pair of jaws 24 that project distally from the distal end of the body 22. The jaws 24 are spaced apart to define a tissue receiving cavity 26 therebetween.

The jaws 24 can have a variety of configurations. For example, as depicted in FIG. 2, the distal ends of the jaws 24 can be outwardly flared so as to increase the size of the distal opening of the tissue receiving area 26. Such a configuration can allow for tissue to be more easily drawn into the tissue receiving area 26. Alternatively or in addition, the inner surfaces of the jaws 24 facing the tissue receiving cavity 26 can have a concave shape to further increase the volume of tissue that can be disposed between the jaws 24. Though the jaws 24 are shown as being substantially parallel to one another, the distance between the jaws 24 can also vary along their length. For example, the distance between the jaws 24 can decrease proximally so as to press together the adjacent layers of tissue that form the tissue fold as the tissue is drawn down between the jaws 24.

As will be appreciated by a person skilled in the art, the jaws 24 can be integral with or fixedly or removably coupled to the body 22 of the end effector 20. Further, though FIG. 2 depicts the jaws 24 coupled to the body 22 such that the jaws 24 maintain a fixed position relative to each other and the body 22, the first and second jaws can alternatively be movably coupled to the end effector 20 such that the jaws can move relative to the end effector 20 and/or one another, as will be described below. As shown in FIG. 2, the fixed distance between the jaws 24 can help separate the cavity wall from surrounding organs as the cavity wall 6 is drawn into the tissue receiving cavity 26. By excluding adjacent organs, the jaws 24 can reduce the risk of adjacent organs being damaged or fastened as part of the tissue fold 8.

As will be appreciated by a person skilled in the art, the jaws can also have a variety of shapes, sizes, and lengths depending on the procedure to be performed. The length of the jaws 24 and the distance between the jaws 24 can vary in order to maximize the depth of the tissue fold. In one aspect, the jaws 24 define a tissue receiving cavity 26 having a length equal to or greater than the desired depth of the tissue fold, such that a tissue fold can be formed and secured through a single tissue acquisition, eliminating the need for multiple tissue bites in order to achieve the desired tissue fold depth.

Controls can also be provided for positioning and/or articulating the end effector 20 and can have a variety of configurations. For example, as shown in FIG. 2, the controls can be in the form of a plurality of control rods 27 connected to the body 22 of the end effector 20. By way of non-limiting example, a pair of control rods can be attached on opposite sides of body 22, while a third rod can be connected to a centralized point at the base of the end effector 20. The rods 27 can articulate the end effector 20 in a range of about −90° to 90° from a linear configuration in which the jaws 24 extend parallel to the longitudinal axis of the elongate shaft 12. The rods 27 can extend proximally through the overtube 2 to an externally controllable actuating assembly to control articulation of the end effector 20.

The surgical device 10 can also include one or more tissue manipulators associated therewith. The tissue manipulator(s) are generally configured to engage tissue to enable tissue to be positioned within the tissue receiving cavity of the end effector and can have a variety of configurations. One having skill in the art will appreciate that the tissue manipulators described herein are exemplary and that any type of tissue manipulator, and even multiple types of tissue manipulators, can be used with any embodiment described herein. Tissue manipulators useful with the devices and methods disclosed herein can be of any type as long as they are effective to grasp tissue in some manner and move it from a first position to a second position or otherwise assist in the reconfiguration of the tissue. By way of non-limiting example, such tissue manipulators include those that are able to grasp tissue by penetrating the tissue (e.g., a corkscrew or a hook) and those that are able to grasp tissue without penetrating the tissue (e.g., vacuum-assisted graspers and pinching devices).

As shown in FIG. 2, the end effector 20 can include a plurality of tissue manipulators 40 that extend distally from the body 22 within the tissue receiving cavity 26. While a single tissue manipulator 40 can be utilized for engaging tissue between the jaws 24, using two or more engagement members 40 can allow the creation of a tissue fold “line,” rather than a “tent.” For example, rather than engaging the cavity wall 6 at a single location, an end effector having multiple tissue manipulators can be effective to grasp tissue at two spaced locations and can create a tissue fold having an increased area in which to place one or more fasteners, thereby decreasing the number of acquisitions or “bites” required for obtaining a fold of the desired length and depth.

The tissue manipulators 40 can include a proximal shaft 42 and a distal tip 44 disposed thereon. The proximal shaft 42 of each tissue manipulator 40 can extend from the tissue receiving cavity 26 proximally through the body 22 of the end effector 20 and can be actuated and/or coupled to an actuator for actuating the tissue manipulator 40. By way of example, the proximal shaft 42 of the tissue manipulators 40 can be coupled to control cables that extend through the elongate shaft 12 to an actuating assembly disposed at the proximal end of the surgical device 10. Through actuation of the control cables, each tissue manipulator 40 can be actuated to rotate about its longitudinal axis, to be axially advanced, and/or to be axially retracted within and beyond the tissue receiving cavity 26. The tissue manipulator shaft 42 can also be flexible, semi-flexible, or rigid, and portions of the shaft 42 can have different flexibilities. For example, a length of the shaft 42 between the distal tip 44 and the body 22 of the end effector 20 can be semi-flexible to allow the tissue manipulator 40 to bend outward as the distal tip 44 penetrates tissue, and retract back between the jaws 24 as the tissue manipulator 40 is axially retracted, to pull the acquired tissue within the tissue receiving cavity 26.

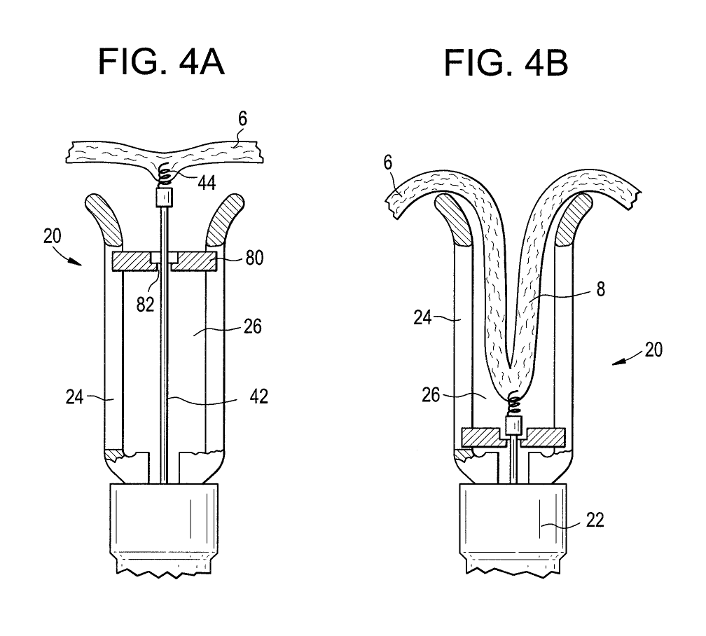

The distal tip of the tissue manipulator can also have a variety of configurations to enable the tissue manipulator to engage, penetrate, and/or grip tissue, and retain the engagement with the tissue as the tissue fold is formed within the tissue receiving cavity. For example, as best shown in FIGS. 4A and 4B, each of the distal tips 44 of the tissue manipulators 40 can be of a corkscrew design to allow the distal tip 44 to be “drilled” into tissue. The corkscrew, for example, can be a helical, coring needle having a rounded end with sharpened edges. As will be appreciated by a person skilled in the art, the size of the distal tips 44 can vary depending upon the desired tissue pulling strength.

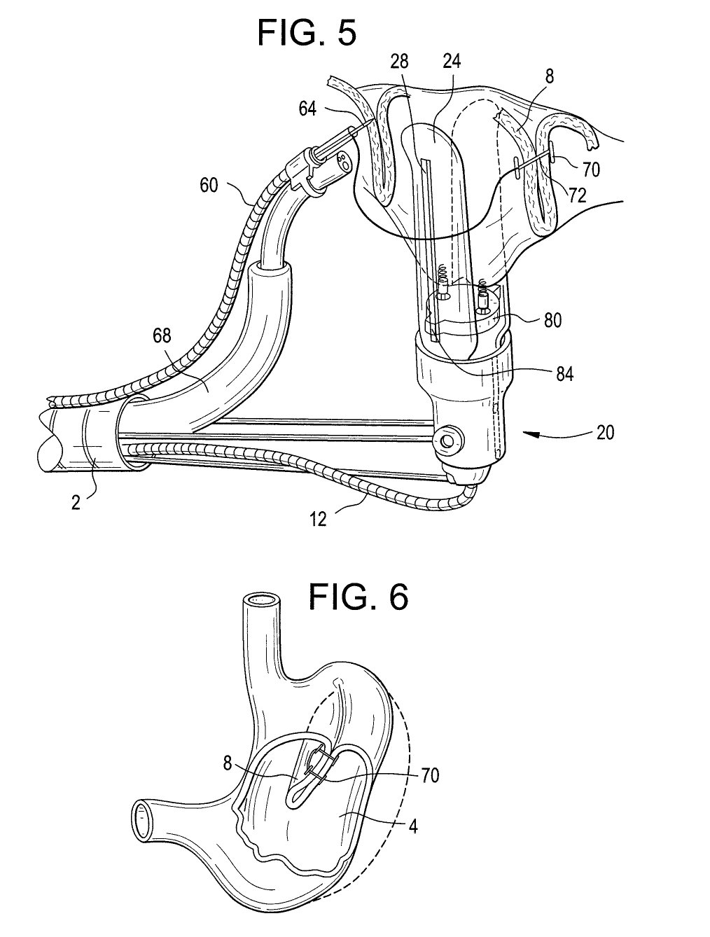

The surgical instrument can also include a stabilization element for controlling the lateral displacement and/or flexion of the tissue manipulator. Additionally, the stabilization element can be effective to support the end effector (e.g., maintaining the tissue receiving cavity during operation). For example, as shown in FIGS. 4A, 4B, and 5, the stabilization element can be a housing 80 located within the tissue receiving cavity 26. The housing 80 can include one or more radially spaced bores 82 through which a shaft 42 of the tissue manipulators 40 can extend. The housing 80 can be movably coupled to the jaws 26. For example, the housing 80 can include radially extending flanges 84 that are configured to slide within a track 28 (e.g., a longitudinally extending slot) formed at least partially within the inner surfaces of the jaws 24. Accordingly, as the tissue manipulator 40 is retracted within the tissue receiving cavity 26, the shaft 42 and distal tip 44 of the tissue manipulator 40 can move proximally relative to the jaws 24 until the distal tip 44 abuts a distal surface of the stabilization element 80. Further proximal movement of the tissue manipulator 40 can cause proximal movement of the housing 80 relative to the jaws 24 as the flanges 84 of the housing 80 slide through the tracks 28. Such a configuration can thereby stabilize the tissue manipulators 40 and reduce their lateral displacement throughout the formation of the tissue fold 8. In one aspect, the axial depth of the housing 80 can be minimized in order to maximize the obtainable depth of the tissue fold 8. The housing 80 and or body 22 of the end effector 20 can also include a mating feature to enable the housing 80 to recess fully into the body 22 of the end effector 20 at the proximal end of the housing's reciprocal path, in order to keep the guide from impinging on the available tissue fold depth.

The surgical device 10 can also be associated with a fastener that is configured to secure adjacent layers of tissue that form the tissue fold. While FIG. 2 depicts the surgical device 10 associated with a fastener 60 for applying T-Tag type suture anchors, a person skilled in the art will appreciate that any tissue fastener, or technique for fastening tissue, known in the art and modified in accord with the teachings herein can be used to more permanently secure a tissue fold. In general, the tissue anchors deployed by the fastener can easily be placed into or through tissue, but can be reconfigured following deployment to an altered configuration in which at least one dimension of the tissue anchor is sufficiently large to maintain the tissue anchor in place. Various tissue anchors which are suitable for securing the tissue folds include, but are not limited to, simple suture knots, reconfigurable “basket”-type anchors (which generally comprise a number of configurable struts or legs extending between two collars or support members), and linear anchors (elongate anchors which are configured to fold or become compressed into a bowed or expanded configuration). Additionally, as will be discussed in detail below, the fastener can be configured to deliver energy to the tissue fold to secure adjacent layers of tissue that form the tissue fold, alternatively or in addition, to the application of a mechanical tissue anchor.

With specific reference to FIG. 3, the fastener associated with the surgical instrument 10 can be a suture anchor deployment device 60 configured to deploy tissue anchors 70 through the adjacent layers of tissue that form the tissue fold 8. An elongated, tubular housing 62 can extend distally from outside the patient and can have a sufficient length to enable the delivery of the suture anchor deployment device 60 through a natural orifice (e.g., the oral cavity) or surgical opening. The suture anchor deployment device 60 can also include a handle and triggering assembly (not shown) remotely operable from outside the body for controlling the discharge of the tissue anchors 70. The suture anchor deployment device 60 can also include a needle 64 having a slotted lumen that extends proximally from the sharpened tip through the suture anchor deployment device 60 for retaining the tissue anchors 70. The needle 64 can retain and deploy one or more tissue anchors 70, with the particular number of anchors loaded into the needle depending on the selected deployment scheme. For example, the tissue anchors 70 can be stacked such that the suture 72 from each tissue anchor 70 exits the tissue anchor 70 near the midsection and perpendicular to the axis of the tissue anchor 70 and is connected to the subsequent tissue anchor 70. The tissue anchors 70 and needle 64 can be aligned so that the suture 72 from the tissue anchors 70 also passes through the needle 64. After the tissue anchors 70 are deployed through the tissue fold 8, the suture 72 can be tightened to pull the tissue anchors 70 together on opposite sides of the tissue fold to cinch the tissue fold and draw the adjacent serosal layer within the tissue fold together. Exemplary suture anchor deployment devices, tissue anchors, and methods of tissue apposition using these devices are described in more detail in U.S. Patent Publication No. 2009/0024163 entitled “Hybrid Endoscopic/Laparoscopic Method for forming Serosa-to-Serosa Plications in a Gastric Cavity,” filed on Jul. 18, 2007, which is hereby incorporated herein in its entirety.

Again referring to FIG. 2, the suture anchor deployment device 60 can optionally be coupled to an optical endoscope 68. For example, the suture anchor deployment device 60 can be coupled adjacent the distal end of the optical endoscope 68 by a ring retainer 66 such that the suture anchor delivery device 60 can move in conjunction with the optical endoscope 68. By attaching the suture anchor deployment device 60 to the optical endoscope 68, the suture anchor deployment device 60 can move separately from and relative to the end effector 20 such that the suture anchor deployment device 60 can be positioned at multiple locations and angles relative to a tissue fold 8 disposed within the tissue receiving cavity 26. The suture anchor deployment device 60 can thus be effective to deploy tissue anchors 70 at multiple locations along the depth or length of a tissue fold 8 without the need to reacquire tissue or to adjust the position of the tissue within the tissue receiving cavity 26. The relative movement between the suture anchor deployment device 60 and the end effector 20 can also enable the suture anchors to be deployed at substantially a 90° angle relative to the tissue fold 8, thereby allowing the tissue anchors 70 to more fully penetrate the tissue layers to provide a more secure tissue fold. Further, by enabling independent articulation of the suture anchor deployment device 60 and the optical endoscope 68 relative to the end effector 20, the user of the system can have improved visualization of the tissue acquisition and fastening. Alternatively or in addition, separate control members can be connected to the suture anchor deployment device 60 for independently articulating the suture anchor deployment device 60. These control members can be operated from external of the patient's body, in conjunction with the trigger assembly of the suture anchor deployment device 60, and can allow the suture anchor deployment device 60 to be articulated in multiple directions to deploy tissue anchors 70 throughout the gastric cavity 4.

Sutures or tissue anchoring devices deployed into and/or through the gastric cavity wall can occasionally fail by being pulled out of the tissue due to contact pressure between the tissue anchor and the secured tissue. This tendency is particularly acute when tension is consistently applied to the tissue anchors by large food volumes caused by patient non-compliance with dietary requirements. To reduce the potential for failure of the tissue anchors, a buttressing device can also be used in conjunction with the tissue anchors. The buttressing device can distribute the load from the tissue anchors across a wider area of the cavity wall, thereby reducing the possibility that tension on the tissue anchor will pull the tissue anchor through the cavity wall. The buttressing device, as well as the tissue anchors themselves, can also be comprised of materials that permit the delivery of therapeutic agents that promote healing, prevent infection, reduce nausea, prevent erosion, induce weight loss, or otherwise provide the patient with a beneficial outcome. The therapeutic agent may be disposed in the implant so as to diffuse or degrade over time in order to advance the treatment or promote healing. Exemplary medicinal agents which can be used with the devices and methods discussed herein are described in more detail in U.S. Pat. No. 7,217,425 entitled “Autologous coatings for implants,” issued on May 15, 2007, which is hereby incorporated herein in its entirety. Exemplary medicinal agents for use with the present device and methods can include, for example, Topomax® brand topiramate, available from Ortho-McNeil Neurologics, Inc. of Titusville, N.J. Topiramate can reduce the need for food and can be used as an adjunct to the surgical procedure. One skilled in the art will appreciate that oral medications can also be used to supplement these effects and that these combination therapies may promote synergies that ultimately greatly increase the efficacy of the surgical procedure.

In use, the surgical device 10 can be inserted into the gastric cavity to form one or more endoluminal plications or tissue folds. As shown in FIG. 1, the end effector 20 can be passed through overtube 2 into the interior of the gastric cavity 4. The optical endoscope 68, with the suture anchor deployment device 60 attached to its distal end, can also be passed transesophageally into the gastric cavity 4 through the overtube 2. As shown in more detail in FIG. 2, the optical endoscope 68 can provide visualization and illumination to enable the surgeon to position the end effector 20 to the desired surgical site within the gastric cavity 4. With the jaws 24 positioned adjacent the cavity wall 6, the tissue manipulator(s) 40 can be advanced from between the jaws 24 into contact with the cavity wall 6. The tissue manipulators 40 can be rotated, for example, while simultaneously being advanced in order to puncture and engage the cavity wall 6.

With reference to the exemplary end effector 20 shown in FIGS. 4A and 4B, after a section of the cavity wall 6 has been acquired by the tissue manipulators 40 (FIG. 4A), the tissue manipulators 40 can be retracted to pull the tissue of the cavity wall 6 into the tissue receiving cavity 26 to form a tissue fold 8, as shown in FIG. 4B. As the tissue manipulators 40 are advanced and retracted within the tissue receiving cavity 26, the housing 80 can also be advanced and retracted through the sliding of the flanges 84 through the track 28 formed in the inner surface of the jaws 24. The corresponding movement of the housing 80 with the tissue manipulators 40 can control the outward flexing of the tissue manipulators 40 and can draw the tissue manipulators 40 back within the tissue receiving cavity 26 as the tissue manipulators 40 are retracted proximally.

As shown in FIG. 5, to fasten the tissue fold 8, the suture anchor deployment device 60 can be positioned relative to the tissue fold 8 retained within the tissue receiving cavity 26 to apply a tissue anchor 70 therethrough. When inserting the tissue anchor 70 through the tissue fold 8, it can be desirable to have as close to normal an angle as possible between the needle 64 and the targeted surface of the tissue fold 8. As shown in FIG. 5, the suture anchor deployment device 60 can be moved in conjunction with the optical endoscope 68 to provide visualization of the targeted location of the tissue anchor 70. Additionally or alternatively, vacuum assist can be applied to draw the targeted fold surface against the face of the tissue anchor deployment device just prior to deployment of the tissue anchor. By way of non-limiting example, the vacuum assist can be applied through a working channel of the optical endoscope 68 or a vacuum tube that can run along the elongated, tubular housing 62. In addition to movement with the optical endoscope 68, the suture anchor deployment device 60 can be separately articulated from the optical endoscope 68 to place the suture anchor deployment device 60 at the desired location.

With further reference to FIGS. 5 and 6, once the suture anchor deployment device 60 has been positioned at the desired location, the needle 64 can be inserted through the tissue fold 8. The tissue anchor 70 can be released through the needle 64 to pass the tissue anchor 70 and its attached suture 72 through the tissue fold 8. After the initial tissue anchor 70 is deployed, the suture anchor deployment device 60 can optionally be moved to additional locations of the tissue fold 8 to further secure the tissue fold 8 with additional tissue anchors 70. After the desired number of tissue anchor(s) 70 have been deployed, the suture material 72 for the tissue anchor(s) 70 can be cinched together, for example, via a working channel of the optical endoscope 68. The number of tissue anchor(s) 70 used to form a tissue fold will depend upon the desired length for the fold and the desired spacing between the tissue anchor(s) 70. In one embodiment, the tissue anchor(s) 70 can be evenly spaced along the length of the desired tissue fold line to form a uniform tissue fold without distortion or bunching. As well, in one aspect, the same number and spacing of suture anchoring devices is deployed into each tissue fold section so that a uniform fold depth is created along the cavity wall 6. The proper relative spacing of the suture anchoring devices can be ascertained, for example, through the optical endoscope 68. Alternatively, a trocar can be inserted into the abdominal wall and used in conjunction with an optical endoscope to visually determine the proper locations for the tissue anchors laparoscopically.

After the tissue fold 8 has been secured, the tissue fold 8 can be released from the tissue receiving cavity 26 by disengaging the tissue manipulators 40 from the cavity wall 6. For example, the tissue manipulators 40 can be extended and/or rotated in the opposite direction from when penetrating the cavity wall 6. The reverse rotation of the tissue manipulators 40 can withdraw the distal tips 44 from the cavity wall 6, thereby releasing the tissue fold 8 from engagement. After the tissue fold 8 is released, the end effector 20 can also be withdrawn such that the secured tissue fold 8 is no longer positioned within the tissue receiving cavity 26.

In one aspect, after a tissue fold 8 has been secured, the end effector 20 can be repositioned adjacent the tissue fold 8. The tissue manipulators 40 can then engage the adjacent section of the cavity wall 6 and pull the additional wall section within the tissue receiving cavity 26. By repositioning the end effector 20 on either side of the previously formed tissue fold, and repeating the tissue engaging and securing steps, the length of the tissue fold 8 can be extended on either side of the initial fold. Additional tissue anchors 70 can be deployed to secure each additional section of the tissue fold 8 until the desired fold length along the cavity wall 6 is achieved. In addition to repeating the plicating procedure to extend the length of the initial fold 8, the end effector 20 can be repositioned to different locations within the gastric cavity 4 to form separate tissue folds at distinct points within the cavity 4.

FIG. 6 depicts a secured tissue fold 8 resulting from process described above. As shown in FIG. 6, a pair of tissue anchors 70 have been deployed in two separate rows to form an involuted fold 8 in the anterior wall of the gastric cavity 4. As the tissue fold 8 is involuted into the interior of the gastric cavity 4, the outer serosal layer of the cavity wall 6 can be drawn into contact with itself in the interior of the tissue fold 8. The tissue anchors 70 can be pulled together by the attached suture 72, and the tension in the suture 72 locked in by a slip knot, knotting element, or other type of suture knot. The tissue anchors 70 placed through the cavity wall 6 can thus maintain the serosa-to-serosa contact within the tissue fold 8 during healing. As can be seen by comparing the dashed line with the solid line boundary of the stomach, the tissue fold 8 can reduce the effective volume of the gastric cavity 4 and can provide many of the advantages mentioned above.

In addition to use of a buttressing device, as discussed above, the opposed serosal layers in contact on the interior of the tissue fold 8 can be treated to reinforce the tissue fold 8. For example, the treatments can promote healing between the contacting serosal surfaces. By way of non-limiting example, the treatment can include abrasion, thermal damage, electrical damage or chemical damage to create scar tissue along the serosal surface. When the treated tissue areas are joined together into a fold, the treatment can induce an earlier and more rapid healing response that may also serve to promote a stronger, more durable bond between the serosal layers. The serosa-to-serosa fold can also be reinforced by injecting a chemical solution into the tissue that forms the tissue fold. The injected solution can toughen the surrounding tissue area to decrease the likelihood that the tissue anchors erode and/or pull through the cavity wall. Suitable chemical solutions (or bulking agents) can include, for example, schlersoants, TGF-beta, keratin, PMMA (polymethyl-methacrylate). Medications that promote healing, such as Vitamin C can also be used to aid in the serosa-to-serosa healing. Such medications can be taken orally, or additionally or alternatively, can be delivered through the tissue anchors or buttress, for example.

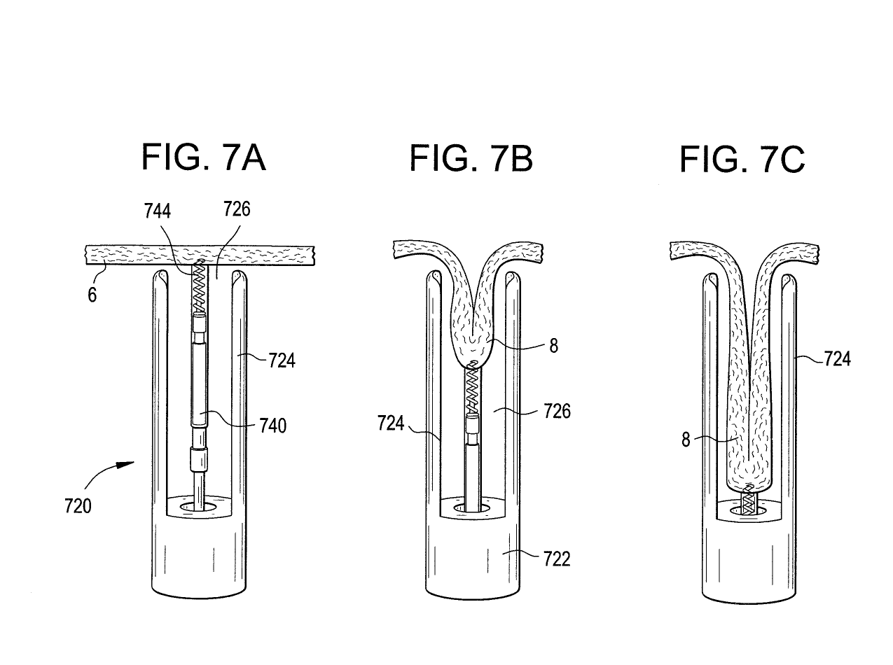

As discussed above, the end effector for use with the surgical instrument of the present invention can have a variety of configurations. For example, FIGS. 7A-7C depict another embodiment of an end effector that can be used to create an endoluminal tissue fold. The end effector 720 is substantially similar to the end effector 20, described above, and includes opposed jaws 724 that can define a tissue receiving cavity 726 therebetween. The opposed jaws 724 can extend distally from the body 722 of the end effector 720. Though the distal ends of the jaws 724 do not flare outward, the distal ends can be substantially rounded to prevent accidental penetration of the cavity wall 6 with the end effector 720. The body 722 of the end effector 720 can also define a bore therethrough that allows an axially extendible and retractable tissue manipulator 740 to extend into the tissue receiving cavity 726.

In use, the end effector 720 can be delivered to the desired surgical site (e.g., within the gastric cavity) and positioned adjacent the cavity wall 6. The tissue manipulator 740 can be extended to engage the cavity wall 6, as shown in FIG. 7A. For example, the tissue manipulator 740 can be axially extended and rotated such that the distal tip 744 of the tissue manipulator 740 is screwed into the cavity wall 6. Following engagement of the cavity wall 6 with the tissue manipulator 740, a tissue fold can be initiated by axially retracting the tissue manipulator 740 with the cavity wall 6 coupled thereto, as shown in FIG. 7B. Though the adjacent layers of the tissue that form the tissue fold 8 can be secured at any point, the depth of the tissue fold can be maximized by fully retracting the tissue manipulator 740 such that the depth of the tissue fold 8 is substantially equal to the length of the tissue receiving cavity 726, as shown in FIG. 7C. As discussed above, once the tissue fold 8 is positioned within the tissue receiving cavity 726, the tissue fold 8 can be secured using a fastener associated with the end effector 720.

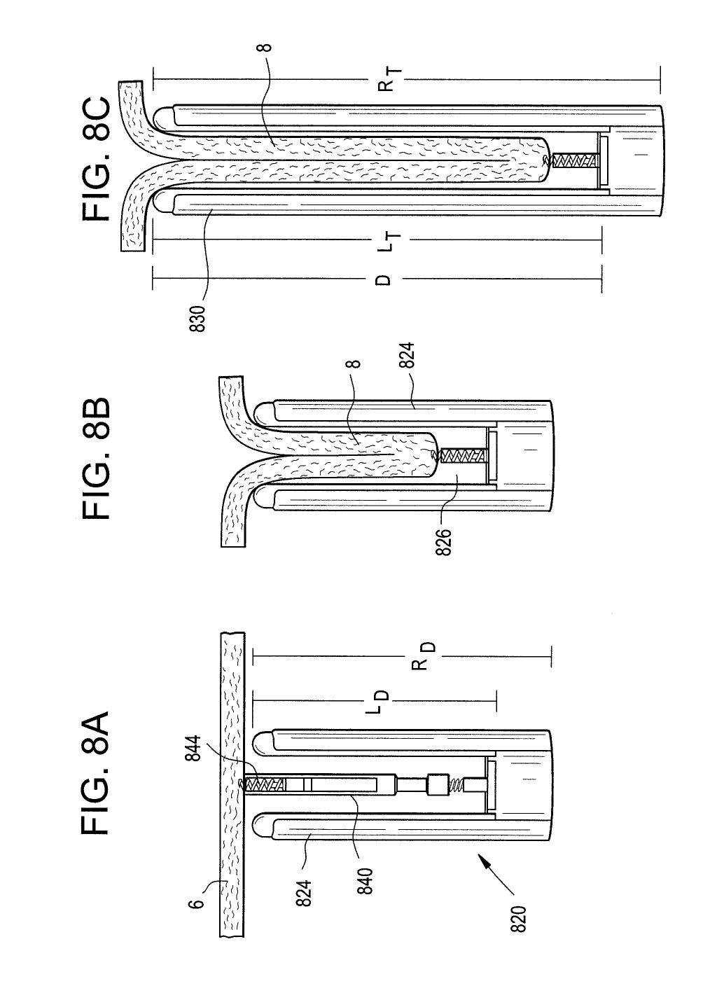

FIGS. 8A-8C depict another embodiment of an end effector that can be used to create an endoluminal tissue fold. The end effector 820 is substantially similar to the end effector 720, depicted in FIGS. 7A-7C, except that the opposed jaws 824 can be extended to increase the length of the tissue receiving cavity 826, thereby enabling additional tissue to be pulled between the jaws 824 to increase the depth of the tissue fold 8. Accordingly, the end effector 820 can have an initial, minimum length as required for delivery to the desired surgical site. After delivery to the desired surgical site (e.g., within the gastric cavity), the jaws 824 can be lengthened. By way of non-limiting example, the jaws 824 can include telescoping extension members 830 movably coupled to the surface of the jaws 824 that face the tissue receiving cavity 826. The telescoping members 830 can also be coupled to an actuator (e.g., a pulley, push rod, etc.) that can be effective to move the telescoping members 830 distally relative to the body 822 of the end effector 820, thereby increasing the length of the jaws 824 and the tissue receiving cavity 826. As the length of the jaws 824 and the tissue receiving cavity 826 is increased, the maximum obtainable depth of the tissue fold 8 can be increased as a greater volume of tissue can be drawn into the tissue receiving cavity 826.

In one aspect, the end effector 820 can be substantially rigid or semi-rigid in order to support the formation of the tissue fold. Accordingly, the rigidity of the end effector 820 can restrict the maximum length of the end effector 820 in order to deliver the end effector 820 to the desired surgical site through a tortuous body lumen of a limited diameter. Thus, as shown in FIG. 8A, the end effector 820 can have a maximum rigid length in the delivery configuration (R_{D}) that can be minimized to ease delivery to the surgical site. After being positioned at the surgical site, the maximum rigid length of the end effector 820 can be increased, for example, by extending the telescoping jaws 824. Upon manipulating the end effector 820, the maximum rigid length of the end effector 820 can be increased to a second maximum rigid length in the treatment configuration (R_{T}), as shown in FIG. 8C. This extension of the telescoping jaws 824 can be effective to create a tissue fold 8 having a depth (D) that is greater than the maximum rigid length of the end effector 820 in the delivery configuration (R_{D}).

In use, the end effector 820 can be delivered to the desired surgical site (e.g., within the gastric cavity) in a delivery configuration, as shown in FIG. 8A. After being positioned adjacent the cavity wall 6, the tissue manipulator 840 can be extended to engage the cavity wall 6. For example, the tissue manipulator 840 can be axially extended and rotated such that the distal tip 844 of the tissue manipulator 840 can be screwed into the cavity wall 6. Following engagement of the cavity wall 6 with the tissue manipulator 840, a tissue fold 8 can be initiated by axially retracting the tissue manipulator 840 within the tissue receiving cavity 826 having a first length (L_{D}) in the delivery configuration, as shown in FIG. 8B. Alternatively, as will be discussed below, the tissue fold can be initiated by extending the telescoping jaws 824. As shown in FIG. 8C, the telescoping jaws 824 can then be extended from their delivery configuration to a treatment configuration in which the length (L_{T}) of the tissue receiving cavity 826 is greater than the first length (L_{D}). Though the adjacent layers of the tissue that form the tissue fold 8 can be secured at any point, the depth of the tissue fold 8 can be maximized by fully retracting the tissue manipulator 840 such that the depth (D) of the tissue fold 8 is substantially equal to the length (L_{T}) of the tissue receiving cavity 826 in the treatment configuration, as shown in FIG. 8C. As discussed above, the tissue fold 8 can then be secured using a fastener associated with the end effector 820. The jaws 824 can be telescoped back to their original length for removal or repositioning of the end effector 820.

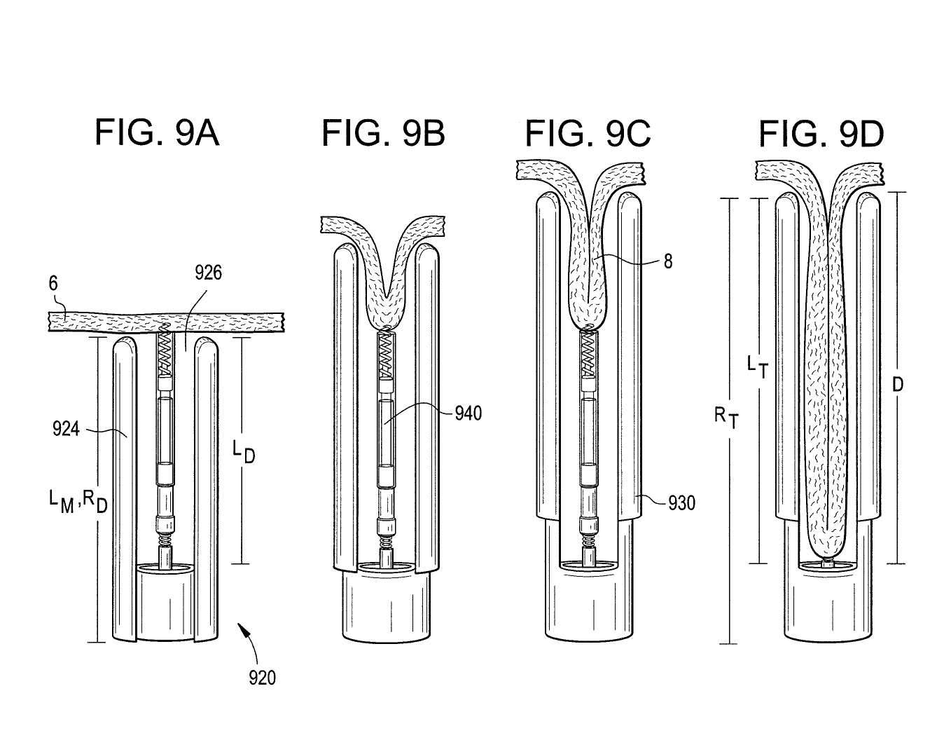

FIGS. 9A-9C depict another embodiment of an end effector that can be used to create an endoluminal tissue fold. The end effector 920 is substantially similar to the end effector 820, depicted in FIGS. 8A-8C, except that the telescoping extension members 930 can be movably coupled to the outer surfaces of the opposed jaws 924. Accordingly, as shown in FIG. 9A, the telescoping members 930 can have a maximum length that is substantially equal to the maximum rigid length of the end effector 920 in the delivery configuration. As above, the telescoping members 930 can be extended to increase the length of the tissue receiving cavity 926 from a first length in the delivery configuration (L_{D}) to a second length in the treatment configuration (L_{T}), thereby increasing the maximum obtainable depth of the tissue fold.

In use, the end effector 920 can form a tissue fold 8 substantially as discussed above in reference to FIGS. 8A-8C. Alternatively, the end effector 920 can be delivered to the desired surgical site (e.g., within the gastric cavity) in a delivery configuration, as shown in FIG. 9A. After being positioned adjacent the cavity wall 6, the tissue manipulator 940 can be extended to engage the cavity wall 6. Following engagement of the cavity wall 6 with the tissue manipulator 940, a tissue fold 8 can be initiated by extending the telescoping jaws 924 from their delivery configuration to a treatment configuration, as shown in FIGS. 9B and 9C. The end effector 920 can have a maximum rigid length (R_{T}) in the treatment configuration that is greater than the maximum rigid length (R_{D}) in the delivery configuration. The tissue manipulator 940 can then be retracted within the tissue receiving cavity 926 having the second length (L_{T}) in the treatment configuration, as shown in FIG. 9D. Though the adjacent layers of the tissue that form the tissue fold 8 can be secured at any point, the depth of the tissue fold 8 can be maximized by fully retracting the tissue manipulator 940 such that the depth (D) of the tissue fold 8 is substantially equal to the length (L_{T}) of the tissue receiving cavity 926 in the treatment configuration, as shown in FIG. 9D. As discussed above, the tissue fold 8 can then be secured using a fastener associated with the end effector 920. The jaws 924 can be telescoped back to their original length for removal or repositioning of the end effector 920.

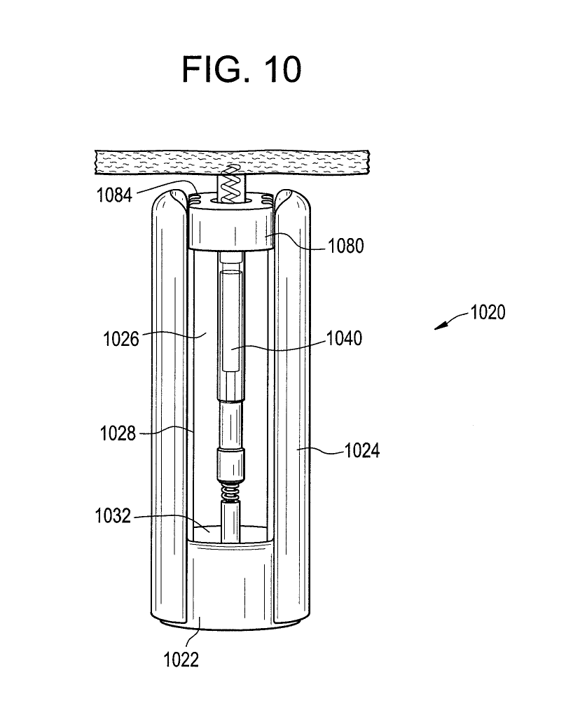

FIG. 10 depicts another embodiment of an end effector that can be used to create an endoluminal tissue fold. The end effector 1020 is substantially similar to the end effector 920 described above with reference to FIGS. 9A-9D, except that the end effector 1020 additionally includes a stabilization element for controlling the lateral displacement and/or flexion of the tissue manipulator. For example, the stabilization element can be a housing 1080 located within the tissue receiving cavity 1026 and movably coupled to the telescoping jaws 1024. The housing 1080 can include a bore through which a portion of the tissue manipulator 1040 can extend. As will be appreciated by a person skilled in the art, the housing 1080 can be coupled to the tissue manipulator 1040 such that it is movable with and/or independent of the tissue manipulator 1040. Additionally, the housing 1080 can be movably engaged with the telescoping jaws 1024 through the cooperation of features formed on the housing 1080 and telescoping jaws 1024. For example, the housing 1080 can include one or more grooves 1084 on the surfaces facing the telescoping jaws 1024. The telescoping jaws 1024 can also include a corresponding number of ribs 1028 that extend longitudinally along the inner surface of the opposed jaws 1024. Accordingly, as the tissue manipulator 1040 is retracted within the tissue receiving cavity 1026, the ribs 1028 can create a track upon which the grooves 1084 of the housing 1080 can slide. In one aspect, the axial depth of the housing 1080 is minimized in order to maximize the obtainable tissue fold depth. The body 1022 of the end effector 1020 can also include a recess 1032 to enable the housing 1080 to recess fully into the body 1022 of the end effector 1020 at the proximal end of the housing's reciprocal path.

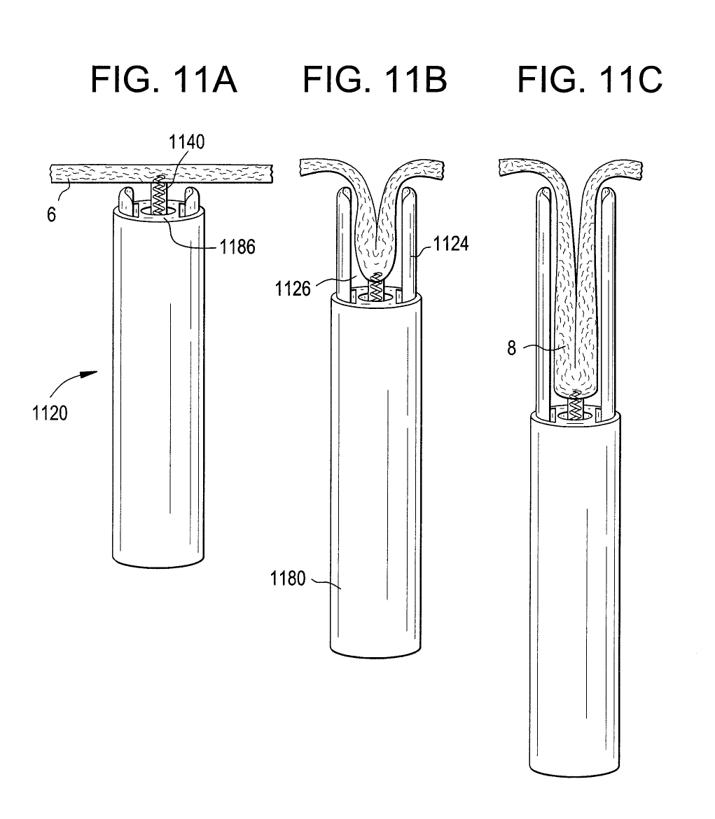

FIGS. 11A-11C depict another embodiment of an end effector that can be used to create an endoluminal tissue fold. The end effector 1120 is substantially similar to the end effector 720 described above with reference to FIGS. 7A-7C, except that the end effector 1120 additionally includes a stabilization element for controlling the lateral displacement and/or flexion of the tissue manipulator. As shown in FIGS. 11A-11C, the stabilization element can be a sheath 1180 disposed substantially around the end effector 1120 and movably coupled thereto. The sheath 1180 can define a lumen through which the end effector 1120 can be disposed and a distal surface 1186 through which the opposed jaws 1124 and the tissue manipulator 1140 can extend. Additionally, the sheath 1180 can be coupled to the tissue manipulator 1140 such that movement of the sheath 1180 can be effective to cause corresponding movement of the tissue manipulator 1140 and vice versa. Thus, the end effector 1120 can be delivered to the surgical site in a delivery configuration with the sheath 1180 disposed therearound, as shown in FIG. 11A. Accordingly, the jaws 1124 of the end effector 1120 can be disposed substantially within the sheath 1180 such that the tissue receiving cavity 1126 has a length in the delivery configuration of substantially zero. That is, until the sheath 1180 is proximally retracted, the jaws 1124 are within the sheath 1180 such that there is not a space within the end effector 1120 in which to receive tissue. After engaging the cavity wall 6 with the tissue manipulator 1140, a tissue fold 8 can be initiated by manipulating the end effector 1120 by proximally retracting the sheath 1180 and the tissue manipulator 1140 such that the cavity wall 6 is pulled into the tissue receiving cavity 1126 between the exposed jaws 1124 of the end effector, as shown in FIG. 11B. Though the adjacent layers of the tissue that form the tissue fold 8 can be secured at any point, the depth of the tissue fold 8 is typically maximized by fully retracting the sheath 1180 and the tissue manipulator 1140 until the tissue manipulator 1140 and sheath 1180 are in their fully retracted position, as shown in FIG. 11C. A person skilled in the art will appreciate that the end effector 1120 can also include additional features described herein. For example, the jaws 1124 of the end effector 1120 can be configured to extend from the distal end of the sheath 1180 as discussed above with respect to FIGS. 8A-8C and 9A-9D.

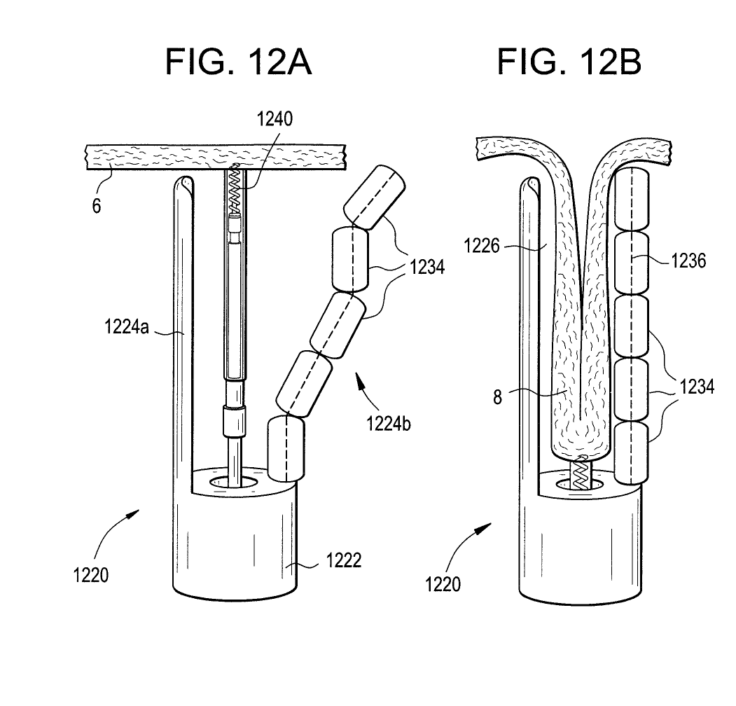

FIGS. 12A and 12B depict another embodiment of an end effector effective to create an endoluminal tissue fold. The end effector 1220 can include a body 1222 having first and second arms 1224a, 1224b extending distally therefrom. The first and second arms 1224a, 1224b can be coupled to the distal end of the body 1222 of the end effector 1220, or alternatively, can extend through the body 1222 of the end effector 1220, e.g., via a bore formed within the body 1222. The first and second arms 1224a, 1224b can also be effective to define a tissue receiving cavity 1226 therebetween. As discussed above, the end effector 1220 can include a tissue manipulator 1240 associated therewith that is configured to engage tissue within the tissue receiving cavity 1226.

The first and second arms 1224a, 1224b of the end effector 1220 can have a variety of configurations, but as shown in FIG. 12A, the first arm 1224a can be substantially similar to one of the arms 24 depicted in FIGS. 4A-4C. That is, the first arm 1224a can be substantially rigid and fixedly coupled to the body 1222 of the end effector 1220 such that the first arm 1224a cannot move relative to the body 1222 of the end effector 1220. The second arm 1224b can be a segmented arm that is formed from a plurality of connected link segments 1234. The connected link segments 1234 can be coupled to one another such that the segmented arm 1224b can be moved between a flexible configuration as shown in FIG. 12A, and a rigid configuration as shown in FIG. 12B. In the flexible configuration, the plurality of connected link segments 1234 can be coupled such that adjacent segments 1234 can move relative to one another. By way of non-limiting example, the plurality of connected link segments 1234 can be coupled together via a joint, such as a flexible portion, hinge, or spring. In the rigid configuration, however, the connected link segments 1234 can be coupled such that link segments 1234 form a rigid segmented arm, such as in a linear configuration, as depicted in FIG. 12B.

The segmented arm 1224b can also include an actuator configured to move the segmented arm 1224b from the flexible configuration to the rigid configuration. For example, the actuator can be in the form of a flexible cable 1236 that can pass through each of the connected link segments 1234. The cable 1236 can extend proximally from the most distal link segment 1234 to outside the patient's body to allow the user to tension the cable 1236. By actuating the cable 1236, the connected link segments 1234 can be pulled together such that the connected link segments 1234 are substantially fixed relative to one another, thereby forming a rigid segmented arm 1224b. Alternatively, the actuator can be in the form of a rigid shaft that can be inserted through the connected link segments 1234 such that the segmented arm 1224b assumes the rigid configuration.

In use, the end effector 1220 can be delivered to the desired surgical site (e.g., within the gastric cavity) and positioned adjacent the cavity wall 6 with the segmented arm 1224b in the delivery configuration as shown in FIG. 12A. Because the connected link segments 1234 can be flexibly connected in the delivery configuration, the segmented arm can pass more easily through the tortuous body lumen than a rigid member of the same length. The tissue manipulator 1240 can then be extended to engage the cavity wall 6. Following engagement of the cavity wall 6 with the tissue manipulator 1240, a tissue fold 8 can be initiated by axially retracting the tissue manipulator 1240 with the cavity wall 6 coupled thereto. Before, during, and/or after manipulating the tissue by retracting the tissue manipulator 1240 with the cavity wall 6 coupled thereto, the end effector 1220 can be moved from the delivery configuration to the treatment configuration. For example, the cable 1236 can be actuated to rigidize the segmented arm 1224b such that the connected link segments 1234 become substantially fixed relative to one another. As the cable 1236 is tensioned, the plurality of connected linked segments 1234 can align such that the segmented arm becomes substantially rigid and parallel with the first arm 1224a, as shown in FIG. 12B. As the segmented arm moves into the treatment configuration, the segmented arm 1224b can capture the cavity wall 6 engaged with the tissue manipulator 1240 such that a tissue fold 8 is formed in the tissue receiving cavity 1226 defined by the first arm 1224a and the rigid segmented arm 1224b in the treatment configuration. The depth of the tissue fold 8 is typically maximized by fully retracting the tissue manipulator 1240 such that the depth of the tissue fold 8 is substantially equal to the length of the tissue receiving cavity 1226, as shown in FIG. 12B. As discussed above, once the tissue fold 8 is positioned within the tissue receiving cavity 1226, the tissue fold 8 can be secured using a fastener associated with the end effector 1220.

Although only the second arm 1224b is depicted as being segmented in FIGS. 12A and 12B, both the first and second arms 1224a, 1224b can be segmented. Accordingly, the rigid length of the end effector 1220 can be defined as the maximum dimension of the longest rigid segment of the end effector 1220 in the delivery configuration (i.e., wherein both of the segmented arms have a rigid length less than their full length due to the ability of the connected link segments 1234 to flex relative to one another). Accordingly, the length of the tissue receiving cavity 1226 between the segmented arms 1224a, 1224b in the treatment configuration, and thus the depth of the tissue fold 8, can be greater than the maximum rigid length of the end effector 1220 in the delivery configuration.

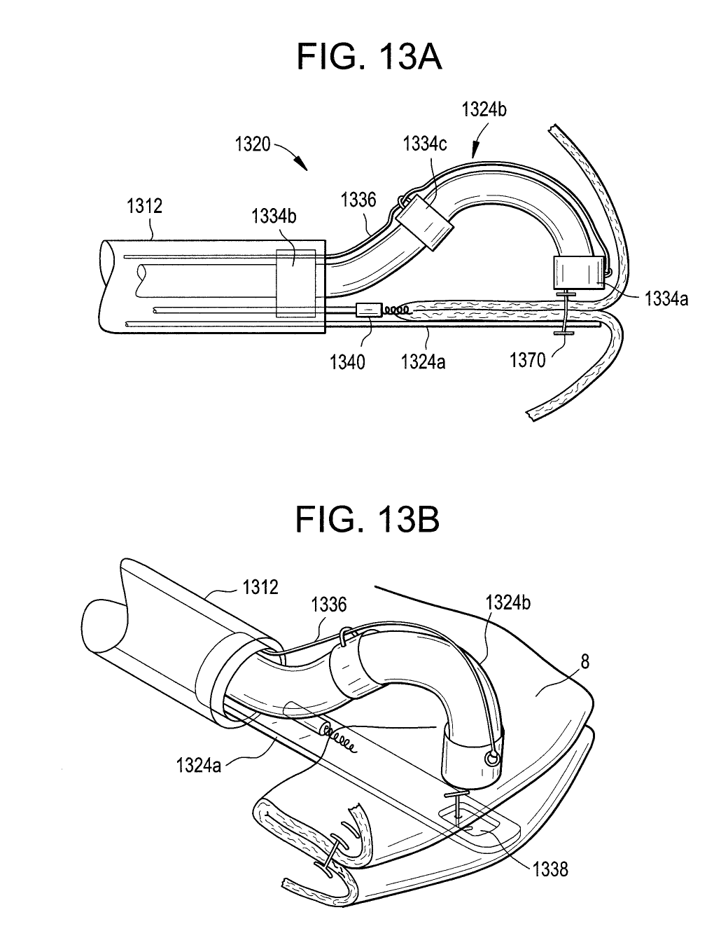

FIGS. 13A and 13B depict another embodiment of a surgical device for forming an endoluminal tissue fold. As shown in FIGS. 13A and 13B, the surgical device can include an end effector 1320 having a first arm 1324a and a steerable arm 1324b. The first arm 1324a can be a flat, longitudinally extending arm that can be configured to extend distally from the distal end of the elongate shaft 1312. The first arm 1324a can be made of a material to resist deflection, or can be composed of a shape memory material such as, for example, Nitinol, which can expand outward as the arm is extended distally. At least one tissue manipulator 1340 can also extend distally from the elongate shaft 1312 for acquiring and/or engaging tissue of the cavity wall 6. As in previous embodiments, the tissue manipulator 1340 can be actuated to advance, retract and rotate relative to the first arm 1324a, in order to acquire and draw tissue proximally and against the first arm 1324a.

The end effector 1320 can also include a steerable arm 1324b that can be used in conjunction with the first arm 1324a to manipulate and stabilize the tissue to form a tissue fold 8. The steerable arm 1324b can be steered within the gastric cavity 4 via a cable 1336 that can be attached at one or more points of the steerable arm 1324b. The cable 1336 can provide support and/or control of the steerable arm 1336 during the formation of a tissue fold 8 within a tissue receiving cavity 1326 between the steerable arm 1324b and the fixed arm 1324a and/or during deployment of the tissue anchor 1370 through the tissue fold 8. The distal end of the cable 1336 can be attached to the distal end of the steerable arm 1324b, while the proximal end of the cable 1336 can extend through the shaft 1312 to provide control of the steerable arm 1324b external of the patient.

As will be appreciated by a person skilled in the art, the cable 1336 can be coupled to the endoscope in any manner known in the art. As shown, the cable 1336 can be attached to the steerable arm 1324b by elastomeric connectors 1334a-c. The connectors 1334a-c can be disposed around the steerable arm 1324b, for example, at a plurality of locations to facilitate control of the steerable arm 1324b. For example, as shown in FIG. 13A, a first connector 1334a can be disposed around the distal end of the steerable arm 1324b, a second connector 1334b can be disposed around the steerable arm adjacent the distal end of the elongate shaft 1312, and a third connector 1334c can be disposed around the steerable arm 1324b at a location between the first connector 1334a and the second connector 1334b. The cable 1336 can extend between the connectors 1334a-c and proximally through the elongate shaft 1312 to enable the user to tension the cable 1336 to enable the steerable arm 1324b to assume an “S” configuration. This “S” configuration in conjunction with the steering of the distal end of the steerable arm 1324b enables the steerable arm 1324b to abut the tissue fold 8 at substantially at a normal angle. The steerable arm 1324b can assume the “S” configuration, for example, by drawing the cable 1336 taut between the connectors 1334a-c.

The connectors 1334a-c can be connected to the steerable arm 1324b prior to insertion of the end effector 1320 into the gastric cavity 4, and they can include a tear away feature for removing the connectors 1334a-c from the endoscope following completion of the procedure. Connectors (and the tear away feature) for use with the present invention are described in greater detail in US Patent Application Publication Number 2008/0103357, entitled “Attachment Apparatus for an Endoscope,” published May 1, 2008, which is hereby incorporated herein in its entirety by reference.

The cable 1336 and connectors 1334a-c provide only one possible means for flexing the steerable arm 1324b into an “S” shape for coordinating with the first arm 1324a to form the tissue fold 8. As will be appreciated by one skilled in the art, a number of different types of methods also exist for similarly controlling the steerable arm 1324b into a similar configuration. For example, the placement of various objects, including, among others, a balloon, wedge or hinge, between the steerable arm 1324b and fixed arm 1324a for pushing the steerable arm 1324b away from the first arm 1324a proximal to the distal opening of the steerable arm 1324b.

Additionally, a working channel extending through the steerable arm 1324b can allow a suture anchor deployment device (not shown) to pass through the steerable arm 1324b. A fastener, e.g. the suture anchor deployment device 60 described above with reference to FIGS. 1-6, can extend through the steerable arm 1324b such that when the distal end of the steerable arm 1324b abuts the tissue fold 8 at a substantially normal angle, a tissue anchor 1370 can be deployed to fasten the tissue fold 8.

The distal end of the first arm 1324a can be closed, such as by a backstop or membrane, to prevent the tissue anchor 1370 from penetrating through the first arm 1324a upon deployment. Alternatively, as shown in FIG. 13B, the distal end of the first arm 1324b can include an opening 1338 to allow the tissue anchor 1370 to be deployed through the first arm 1324a. In one embodiment, a buttressing material can be included within the opening 1338 so that the tissue anchor 1370 also penetrates the material during its deployment. After penetration of the material by the tissue anchor 1370, the buttressing material can be released from the opening 1338 to provide reinforcement to the tissue fold 8 and/or to prevent failure of the tissue anchor 1370. Additional buttressing material can alternatively or additionally be provided between the distal opening of the steerable arm 1324b and the tissue fold 8 to reinforce the other side of the tissue fold 8.

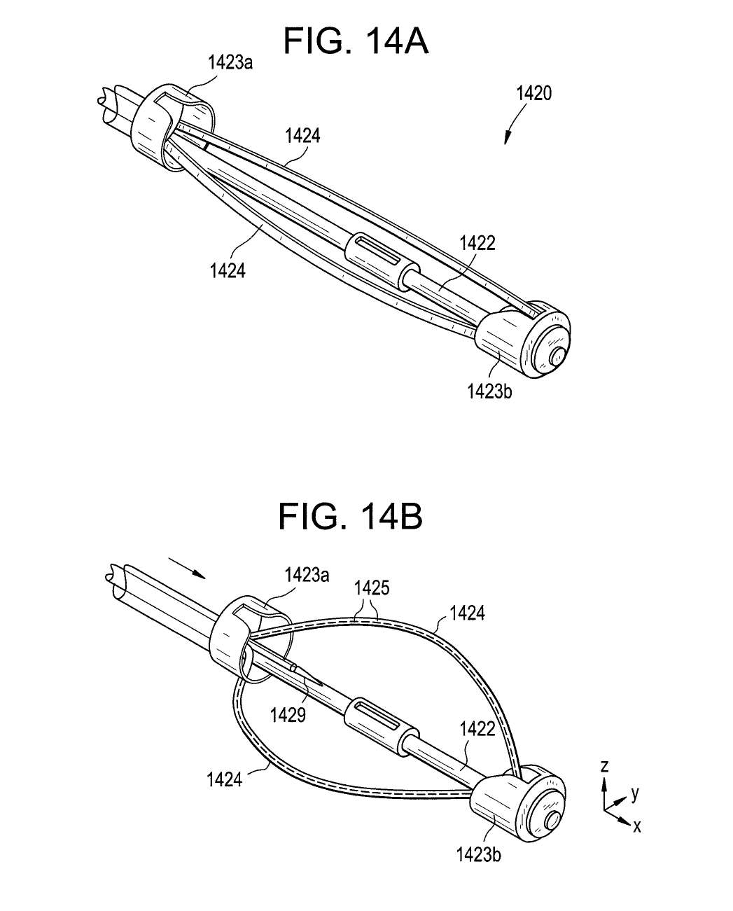

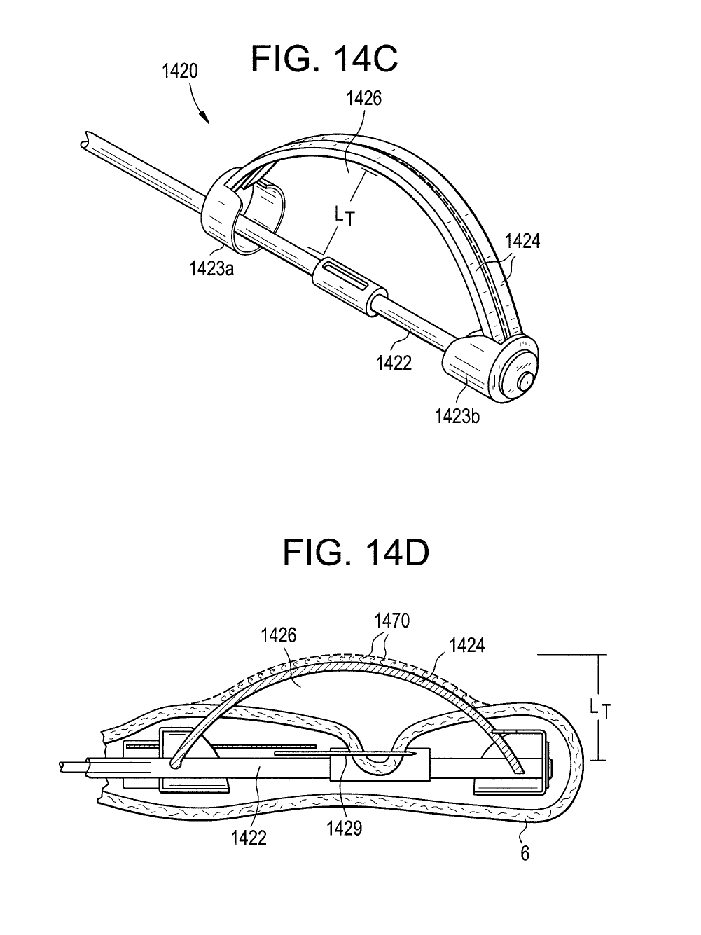

FIGS. 14A-14D depict another embodiment of an end effector for use with a surgical device for forming an endoluminal tissue fold. As shown in FIGS. 14A and 14B, the end effector 1420 can include a pair of arms 1424 that extend radially outward from a tubular body 1422. The end effector 1420 can include end caps 1423a,b disposed on the body 1422 effective to retain the proximal and distal ends of the arms 1424. At least one of the end caps 1423a,b can be movable relative to the body 1422 and/or the other end cap such that the arms 1424 can be reconfigured based on the relative position of the endcaps 1423a,b. For example, the proximal end cap 1423a can be moved (e.g., slid along body 1422) from a first, proximal position, as shown in FIG. 14A, to a second more distal position, as shown in FIG. 14B. By way of non-limiting example, the end cap 1423a can be in the first position during insertion of the end effector 1420 into the gastric cavity 4 such that the arms 1424 assume a low profile configuration in which the arms 1424 are adjacent and substantially parallel to the body 1422, as shown in FIG. 14A. This low profile configuration can ease the passage of the end effector 1420 through an overtube or other transoral delivery device.

Once the end effector 1420 has been introduced to the gastric cavity, for example, the end effector 1420 can be moved into an open, delivery configuration, as shown in FIG. 14B. By way of non-limiting example, the end caps 1423a,b can be compressed, such as by activation of an actuator cable (not shown) that extends through the body 1422, relative to one another such that the arms move from a position proximate to and substantially parallel to the body 1422 to a bowed configuration, as shown in FIG. 14B. In this open, delivery configuration, the arms 1424 can be substantially planar (e.g., substantially on the plane defined by the x-axis and y-axis as shown in FIG. 14B) relative to the body 1422 such that the arms 1424 and body 1422 can be positioned adjacent the wall of the gastric cavity. Thus, in a delivery configuration, the tissue receiving cavity 1426 can have a length (e.g., in the z-axis as shown in FIG. 14B) of substantially zero.

As will be appreciated by a person of skill in the art, the arms 1424 can be formed of any material(s) that allow the arms 1424 to be reconfigured from a low-profile configuration to a bowed configuration, such as by the application of a compressive force. By way of non-limiting example, the arms can be formed from a polymeric material, a shape memory material, and/or metals such as stainless steel. In one embodiment, the arms 1424 can be comprised of a shape memory material that enables the arms 1424 to assist the reconfiguration of the end effector 1420.

An actuating cable can extend through the body 1422, in contact with the distal and proximal ends of the arms 1424 to move the arms between the open, delivery configuration and a closed, tissue engaging configuration, as shown in FIG. 14C. By way of non-limiting example, the end caps 1423a,b can be further compressed, such as by activation of an actuator cable, rotating the arms 1424 from the bowed position in FIG. 14B to the position shown in FIG. 14C through contact with sloped edges of the end caps 1423a,b. A plurality of tissue anchors 1470, such as, for example, staples, T-Tag anchors, nitinol springs, etc. can be disposed on a tissue contacting surface of the arms 1424 or included within the arms 1424. The openings 1425 can be provided in the arms 1424 for releasing the tissue anchors 1470 into a tissue fold 8 formed within the tissue receiving cavity 1426.

The tubular body 1422 can further include an opening 1427 into the interior of the body 1422. A tissue manipulator (not shown) can be configured to extend through the tubular body 1422 and out of the opening 1427 to engage the cavity wall 6, as discussed elsewhere herein. Alternatively or additionally, vacuum pressure can be applied through the opening 1427 to pull and/or retain tissue therein during deployment of the arms 1424. A needle 1429, shown in FIG. 14D, can also extend through the interior of the body 1422. The needle 1429 can have sufficient length for advancing into and penetrating tissue drawn into the opening 1427 in order to hold the tissue stable within the opening 1427. It is conceived that tissue grasping means including but not limited to graspers, corkscrews, hooks, and rollers may secure the tissue in a similar manner as described using needle 1429.

In use, the end effector 1420 can be delivered to the gastric cavity to form one or more endoluminal tissue folds. For example, the end effector can be inserted through a transoral passage in a low-profile configuration, as shown in FIG. 14A. After being introduced into the gastric cavity, the proximal end cap 1423a can be slid distally relative to the distal end cap 1423b such that the arms 1424 assume the open, delivery configuration shown in FIG. 14B. With the arms 1424 in the open configuration, the tissue manipulator can be introduced into the gastric cavity 4 through the opening 1427. The tissue manipulator can be actuated to engage the cavity wall 6 and subsequently retracted to draw a portion of the cavity wall 6 into the opening 1427. Vacuum pressure can be applied to further draw the tissue layers into the opening 1427, as shown in FIG. 14D. Additionally, the needle 1429 can be advanced into and through the tissue drawn into the opening 1427 to lock the tissue therein. With the tissue cavity wall 6 secured within the opening 1427, the arms 1424 can be moved from the open, delivery configuration to the closed configuration as shown in FIG. 14C to thereby capture a section of the cavity wall 6 within the tissue receiving cavity 1426. For example, as the arms move from the substantially planar delivery configuration, the arms 1424 can swing in an arc through the gastric cavity 4 and engage a large, circular section of the cavity wall 6. As the arms 1424 swing together, the arms 1424 can draw the wall section together into a tissue fold 8 within the tissue receiving cavity 1426. Tissue anchors 1470 can be then be deployed into the tissue fold 8 retained between the arms 1424 to secure the adjacent layers of tissue that form the tissue fold 8. For example, the adjacent serosal layers on the interior of the tissue fold can be secured together. As discussed above, the end caps 1423a,b can be moved relative to one another (e.g., compressed and expanded) in the open, delivery configuration such that the arms 1424 can be configured to maximize the depth of the tissue fold 8. By way of non-limiting example, as the proximal end cap 1423a is moved distally relative to distal end cap 1423b, the length (L_{T}) of the tissue receiving cavity 1426 defined by the arms 1424 in the tissue engaging configuration can be increased. Following the fastening of the tissue fold 8, the arms 1424 can be actuated back to their open, delivery configuration. The needle 1429 can be retracted proximally within the body 1422, and the vacuum disengaged to release the tissue from opening 1427. After the tissue is released from the end effector 1420, the end cap 1423a can be slid proximally relative to the distal end cap 1423b such that the arms 1424 assume a low profile configuration in which the arms 1424 are in longitudinal alignment with the body 1422 such that the end effector 1420 can be withdrawn through the overtube.

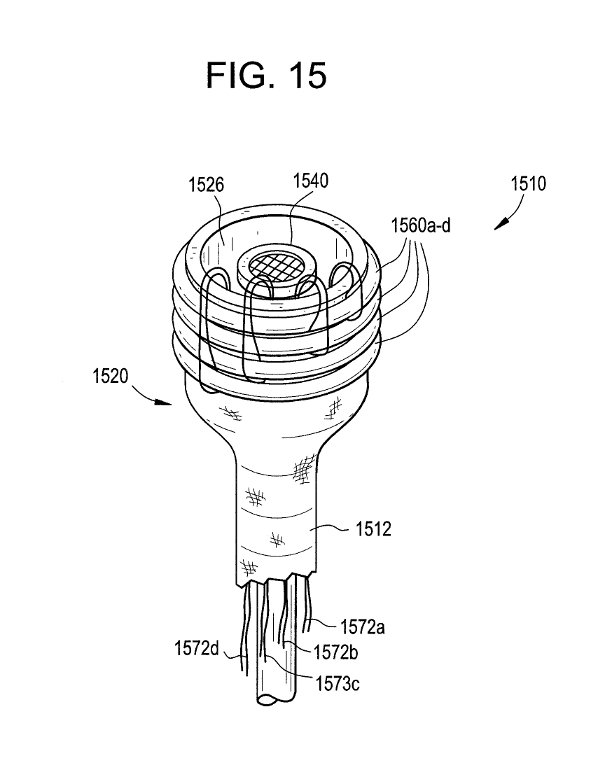

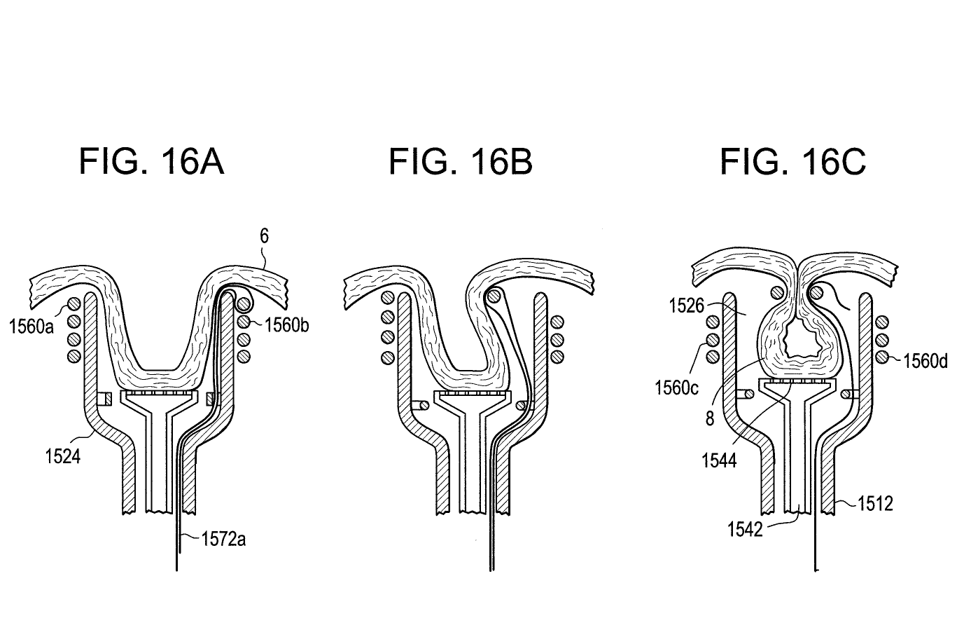

FIGS. 15 and 16A-16C depict another embodiment of a surgical device for forming an endoluminal tissue fold to reduce the volume of the gastric cavity. As shown in FIG. 15, the surgical device 1510 generally includes an end effector 1520 disposed on the distal end of a flexible shaft 1512, a tissue manipulator 1540, and a fastener. As discussed above, end effectors for use in the present invention generally include a tissue receiving cavity 1526 in which a tissue fold can be formed. In the embodiment depicted in FIGS. 15 and 16A-16C, the end effector 1520 can include a flared distal portion 1524 that can define the tissue receiving cavity 1526. Additionally, a fastener in the form of a plurality of elastic rings 1560a-d can be stretched and stacked around the outer circumference of the flared distal portion 1524. Pull strings 1572a-d can be looped through an individual corresponding ring 1560a-d and can extend proximally through the lumen of the elongate shaft 1512 to enable external deployment of the elastic rings 1560a-d.

As discussed above, the tissue manipulator is generally configured to engage tissue to enable tissue to be positioned within the tissue receiving cavity of the end effector and can also have a variety of configurations. As shown, the end effector 1520 can also be associated with a tissue manipulator 1540 that is configured to engage tissue (e.g., the wall of the gastric cavity) to enable the tissue to be positioned within the tissue receiving cavity 1526. The tissue manipulator 1540 generally includes a proximal tubular portion 1542 and a distal opening 1544 through which vacuum pressure can be applied. The vacuum can be effective to draw the cavity wall 6 against the distal opening 1544 of the tissue manipulator 1540. The tissue manipulator 1540 can be in a fixed position relative to the end effector 1520 such that the vacuum is effective to draw the tissue into the tissue receiving cavity 1526. Alternatively, as discussed above, the tissue manipulator 1540 can be configured to axially extend and retract to capture tissue.

As shown in the sequence depicted in FIGS. 16A-16C, the end effector 1520 can be delivered to the gastric cavity to form one or more endoluminal tissue folds. As shown in FIG. 16A, the distal end of the end effector 1520 can be positioned adjacent the desired surgical site. Vacuum pressure applied through the distal end 1544 of the tissue manipulator 1540 can be applied to engage the cavity wall 6, and the tissue manipulator 1540 can be retracted distally within the tissue receiving cavity 1526. Alternatively, the vacuum pressure applied by the tissue manipulator 1540 can be sufficient to simply draw the cavity wall 6 into the tissue receiving cavity 1526. That is, the tissue manipulator 1540 can manipulate the cavity wall 6 simply by drawing the cavity wall 6 into the tissue receiving cavity 1526, without having to extend and/or retract the tissue manipulator 1540. Regardless, when the cavity wall 6 is pulled into the tissue receiving cavity 1526 to a desired depth, the pull string 1572a can be actuated to dislodge the ring 1560a from around the perimeter of the end effector 1520, as shown in FIG. 16B. As the ring 1560a is pulled from around the perimeter of the end effector 1520, the ring 1560a can contract inward due to its elasticity such that the ring 1560a encircles and secures the tissue fold disposed within the tissue receiving cavity 1526 to form a tissue nodule 8, as shown in FIG. 16C. After the tissue nodule 8 is secured, the vacuum pressure of the tissue manipulator 1540 can be discontinued to release the tissue nodule 8 from the end effector 1520. The end effector 1520 can subsequently be moved to another targeted section of the cavity wall 6 to form another nodule by, for example, pulling a second pull string to release the second ring 1560b around a second tissue fold disposed within the tissue receiving cavity 1526 to form a second tissue nodule.

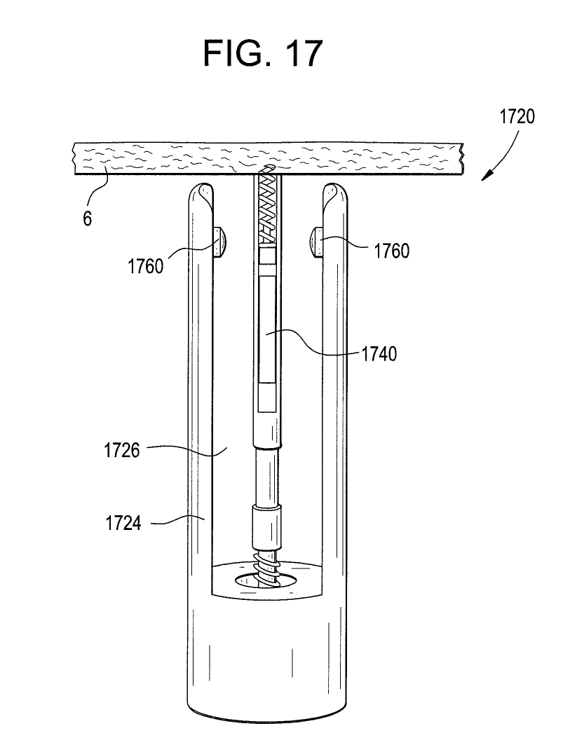

Alternatively or in addition to the fasteners discussed above, the surgical devices for forming an endoluminal tissue fold can be associated with a fastener that includes one or more electrodes configured to deliver energy to secure adjacent layers of tissue that form the tissue fold. FIG. 17 depicts one embodiment of an end effector for forming an endoluminal tissue fold that includes an electrode for applying energy to the tissue fold. The end effector 1720 is substantially similar to the end effector 720 described above with reference to FIGS. 7A-7C except that the end effector 1720 includes two electrodes 1760 (one of which can be an active, energy-delivering electrode and the other of which can be a return electrode) disposed on the tissue contacting surface of the opposed jaws 1724. The electrodes 1760 can be configured to contact the outer surfaces of a tissue fold 8 when the tissue fold 8 is disposed within the tissue receiving cavity 1726. Though the electrodes 1760 are depicted with reference to the end effector 1720, it will be appreciated that any of the embodiments of the surgical device described herein can include electrodes for delivering energy to a tissue fold.

As will be appreciated by a person skilled in the art, the electrodes 1760 can have various configurations depending on the energy to be applied. By way of non-limiting example, thermal energy, electrical energy, acoustic energy (e.g., ultrasonic), and/or radiofrequency (RF) can be applied by the electrode(s) to the tissue fold to bond the opposed serosal layers in contact on the interior of the tissue fold. Further, the skilled artisan will appreciate that the treatment parameters (e.g., power, energy, delivery time, frequency, wave pattern) and the physical parameters of the energy delivery system (e.g., the number, diameter, spacing, and location of the electrodes) can be optimized to secure the adjacent layers of tissue. The skilled artisan will also appreciate that the surgical device can include an energy source operatively coupled to the electrode(s) to activate the one or more electrode(s). By way of example, the energy source can be a battery disposed within the surgical device, or the surgical device can be adapted to couple to an external energy source, such as a generator or an outlet. Further, the surgical device can include a mechanism to activate the delivery of energy by the electrode(s), such as a button or dial.

In use, the end effector 1720 can be delivered to the desired surgical site (e.g., within the gastric cavity) and positioned adjacent the cavity wall 6. As above, following engagement of the cavity wall 6 with the tissue manipulator 1740, the tissue can be manipulated to form a tissue fold within the tissue receiving cavity 1726. With the tissue fold in contact with the electrodes 1760 disposed on a tissue contacting surface of the opposed jaws 1724, energy can be delivered to the tissue fold through one or more electrode(s) 1760. For example, an electrode 1760, operating in either a bipolar or monopolar mode, can deliver RF energy to the tissue fold such that the adjacent layers of the tissue that form the tissue fold are secured.

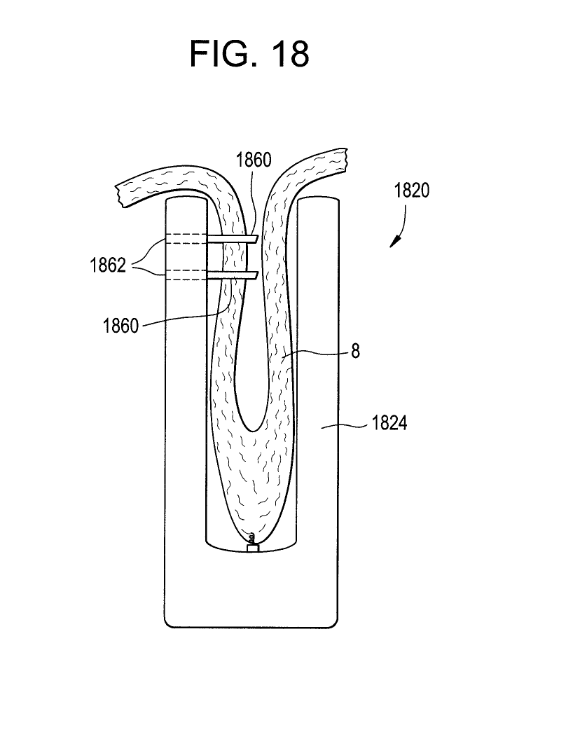

FIG. 18 depicts another embodiment of an end effector for forming an endoluminal tissue fold that includes an electrode for applying energy to the tissue fold. The end effector 1820 is substantially similar to the end effector 1720 described above with reference to FIG. 17, excluding electrodes 1760. Instead the end effector 1820 is associated with electrodes 1860 that can be movable between a first position, in which the electrodes 1860 are substantially disposed within bores 1862 through one of the opposed jaws 1824, to a second position in which at least portions of the electrodes 1860 extend into the tissue receiving cavity 1826. As shown in FIG. 18, the electrodes 1860 can be configured to penetrate at least partially through the adjacent layers of tissue that form the tissue fold 8 disposed within the tissue receiving cavity 1826. By way of example, the electrodes 1860 can be in the form of a needle configured to pierce the tissue that forms the tissue fold 8. Although the needle electrodes 1860 are depicted as being inserted into the tissue fold 8 substantially transverse to the central axis of the tissue fold, the electrodes can be configured to be inserted into the tissue fold 8 at any angle.

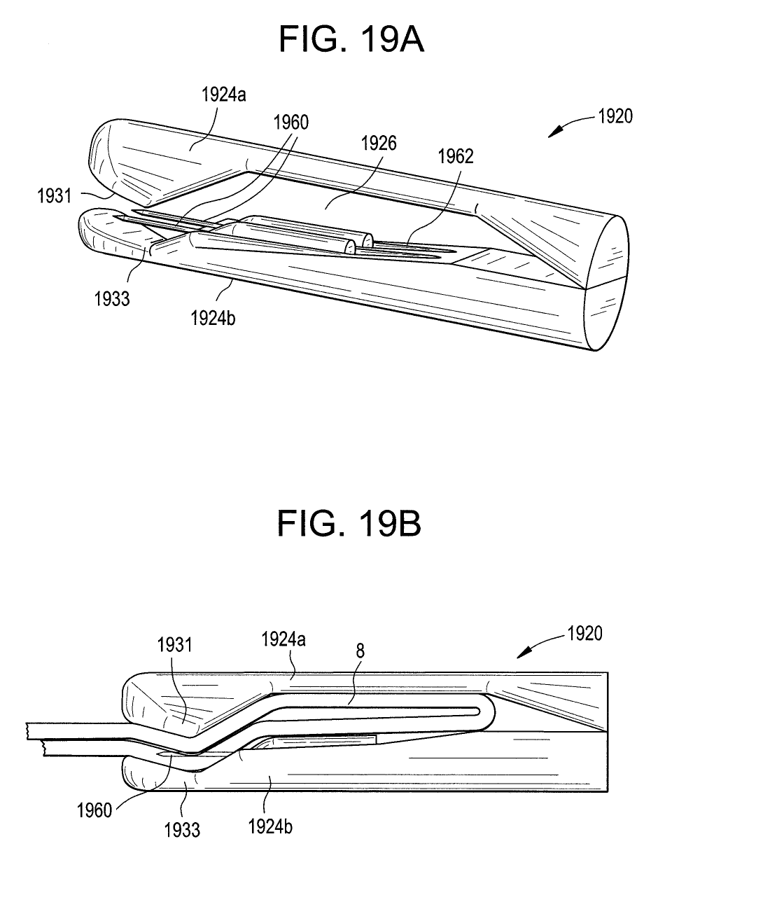

For example, referring now to FIGS. 19A and 19B, the end effector 1920 can include electrodes 1960 configured to be inserted into the tissue fold 8 substantially along the central axis of the tissue fold 8. As shown in FIG. 19, the end effector 1920 can include opposed jaws 1924a,b defining a tissue receiving cavity 1926 therebetween. The end effector 1920 can additionally include a tissue manipulator (not shown) that is configured to engage tissue within the tissue receiving cavity 1926, as described elsewhere herein. The opposed jaws 1924a,b and the tissue receiving cavity 1926 can have a variety of configurations, but as depicted in FIG. 19A, the opposed jaws 1924a,b can be configured to manipulate the tissue fold 8 formed within the tissue receiving cavity 1926 such that at least a portion of the tissue fold 8 is bowed. For example, the first jaw 1924a can include a protrusion 1931 and the second jaw 1924b can include a trough 1933 opposed to the protrusion 1931. Accordingly, a tissue fold 8 disposed within the tissue receiving cavity 1926 can bend around the protrusion 1931 and extend into the trough 1933. The end effector 1920 can also include electrodes 1960 that are substantially disposed within one of the opposed jaws. For example, the second opposed jaw 1924b can include one or more bores 1962 in which the electrodes 1960 can be disposed. The electrodes 1960 can be actuated between a first position, in which the electrodes 1960 are substantially disposed within the bores 1962, to second position in which at least portions of the electrodes 1960 extend from the bores 1962 and into the tissue receiving cavity 1926. As shown in FIG. 19B, actuation of the electrodes 1960 can be effective to extend the electrodes 1960 into the bowed portion of the tissue fold 8 disposed within the tissue receiving cavity 1926 substantially along the longitudinal axis of the tissue fold 8 such that one or both of the layers of tissue that form the tissue fold 8 are penetrated. Accordingly, the electrodes 1960 can apply energy to the interior of the tissue fold 8, for example, to the opposed serosal layers. By way of non-limiting example, the electrodes 1960, operating in either a bipolar or monopolar mode, can deliver RF energy to the tissue fold 8 such that the interior serosal layers of the tissue fold 8 are bonded together.

It is envisioned that reducing the volume of the gastric cavity and/or the preceding devices and procedures for reducing a gastric cavity can be supplemented by the addition of a satiety agent or a derivative thereof or analogs thereof within the digestive system. Such active agents include naturally occurring or synthetic hormones, peptides, neurotransmitters or mimetics thereof, that are capable of directly stimulating the region responsive to the satiety agent (e-g., the duodenum). Specific active agents which are useful include but are not limited to peptide hormones, agonists to peptide hormone receptors, antagonists to peptide hormone receptors, CCK (GenBank Accession No. NP-000720), Bombesin, Gastrin releasing peptide (GRP), glucagon, Enterostatin, Ghrelin, GLP-1 (glucagon-like peptide) (Bojanowska E., 2005, Med. Sci. Monit. 11:RA271-8; BYETTA™ (exenatide)), PYY (le Roux C W., et al., 2005, Endocrinology. 2005 Sep. 15; GenBank Accession No. NP-004 15 I), Oxyntomodulin 15 (OXY, OXM; GenBank Accession No. P01275; Stanley S., et al., 2004, Am. J. Physiol. Gastrointest. Liver Physiol. 286(5): G693), Melanocortin 4 receptor Agonists, Apo IV (naturally occurring apoprotein Qin X, Tso P 2005, Curr Drug Targets. 6(2): 145-51), GI18 177 1X (GSK), anti Ghrelin agents (Kobelt P., Gut. 2005 Jun. 30; SPIEGELMER NOX-B1 I), substances that prevent the acylation of ghreling, PP (Miskowiak J, et al., 1985, Regul. Pept. 12: 231-6) and derivatives and analogs thereof such as CCK-4 (Trp-Met-Asp-Phe), CCK-8 (Asp-Tyr(S03H)-Met-Gly-Trp-Met-Asp-Phe) analogues of CCK), CCK analogs ((Sincalide by Bracco Diagnostics or Squibb Diagnostics), GSK-GW7176, GW 5283, GW7854 and Pfizer PW170292)), CCK receptor agonists (e.g., 1,5-benzodiazepines, PD 170292, SR 146 13 1) and/or activator molecules of the CCK-A receptor (JMV 180; Archer-Lahlou E, et al., 2005, 25 J. Biol. Chem., Vol. 280: 10664-10674), and PYY analogs (e.g., PYY(1-36), PYY(3-36), PYY(9-36), PYY(14-36), PYY(22-36), and PYY(27-36)).

The satiety agent can be introduced by implanting a pump within the body or attaching a pump to the body, and the pump can have an outlet introducing the satiety agent to a portion of the digestive system where the satiety agent is found to be most effective, such as the duodenum. Alternately, the satiety agent can be administered orally in pill form or can be injected into the body. The satiety agent can also be sprayed or poured in liquid form on the interior surface of the digestive system.

A kit may be described containing devices and satiety agents needed to perform a method of plicating a gastric cavity and introducing a satiety agent. The kit can include, for example, an apparatus for reducing the volume of the gastric cavity, a satiety agent, and an introducer for the satiety agent.

It is further envisioned that a procedure for forming a tissue fold within the gastric cavity can be supplemented by surgically altering another portion of the digestive system. For example, one portion of the digestive system can be transposed to another portion of the digestive system. As further example, a surgeon can reduce the volume of the gastric cavity, remove a portion of the ileum, and anastomose the portion of the ileum to reside in-line with a section of the duodenum. The surgeon can then anastomose the open sections of ileum where the transposed portion was removed to recreate a continuous digestive system. A surgeon can use, for example, an Echelon™ Endoscopic Linear Cutter, available from Ethicon Endo-Surgery, in Cincinnati, Ohio, to perform the anastomoses. Without being limited by theory, it is believed that transposing a section of the ileum to the duodenum may reduce a patient's cholesterol and/or increase a patient's lean muscle mass. Satiety agents can also be introduced as described above.

It is further envisioned that the techniques described herein for endoscopic plication formation can be combined with devices and/or methods to implant a duodenal barrier in a gastrointestinal tract containing a volume reduction procedure as described in more detail in U.S. Patent Publication No. 2009/0276055 entitled “Method for Gastric Volume Reduction Surgery,” filed on May 1, 2008, which is hereby incorporated herein in its entirety.

Additional kits can be provided containing devices for performing gastric volume reduction and ileal transposition. For example, a can may contain one or more of the following: a device for gastric volume reduction; a device for transposing the ileum; a satiety agent; and an introducer for the satiety agent.

Further, any and all of the various embodiments of the surgical devices embodiments disclosed herein can be interchangeable with one another as needed. For example, any of the end effectors described above can be associated with a fastener configured to deliver energy to a tissue fold to secure adjacent layers of tissue that form the tissue fold.

Further, as the surgical device is delivered to the desired surgical site, a risk can exist that the device can tear or puncture nearby tissue. Accordingly, in any and all of the embodiments described herein, a safety shield can optionally be included to reduce the risk of tearing or puncture by the surgical device. In general the shield can be of a material that is relatively smooth to allow ease of passage of instruments, but resistant to tearing and puncture. For example, the shield can be formed of silicone, urethane, thermoplastic elastomers, rubber, polyolefins, polyesters, nylons, fluoropolymers, and any other suitable materials known in the art. The shield can be detachable from a surgical access device so it can be used as needed in a particular procedure. The shield can also be integral with the any of the surgical access device embodiments or any of the components described herein. The components themselves can also act as shields.

In any and all of the surgical devices disclosed herein, an engagement and/or release mechanism can be included to allow one component to be separated from another component or to allow one portion of a component to be separated from another portion of a component. For example, a jaw can be separable from the end effector. The engagement or release mechanism can be a latch, switch, c-clamp, tabs, push button, or any other mechanism known in the art that can be configured to release one portion of a device from another.

There are various features that can optionally be included with any and all of the surgical device embodiments disclosed herein. For example, a component of the device, such as the elongate shaft, end effector, tissue manipulator, or fastener, can have one or more lights formed thereon or around a circumference thereof to enable better visualization when inserted within a patient. As will be appreciated, any wavelength of light can be used for various applications, whether visible or invisible. Any number of working channels, suspension members or tethers, seal housings, and seal elements can be included on and/or through the retractor to enable the use of various surgical techniques and devices as needed in a particular procedure. For example, openings and ports can allow for the introduction of pressurized gases, vacuum systems, energy sources such as radiofrequency and ultrasound, irrigation, imaging, etc. As will be appreciated by those skilled in the art, any of these techniques and devices can be removably attachable to the surgical access device and can be exchanged and manipulated as needed.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Typically, the invention described herein will be processed before surgery. First, new or used surgical instruments and access devices are obtained and cleaned, if necessary. The surgical equipment can then be sterilized. Any number of sterilization techniques known to those skilled in the art can be used to sterilize the equipment including beta or gamma radiation, ethylene oxide, steam, and a liquid bath (e.g., cold soak). In one sterilization technique, the equipment is placed in a closed and sealed container, such as a plastic or TYVEK bag. The container and equipment are then placed in a field of radiation that can penetrate the container, such as gamma radiation, x-rays, or high-energy electrons. The radiation kills bacteria on the equipment and in the container. The sterilized equipment can then be stored in the sterile container. The sealed container keeps the equipment sterile until it is opened in the medical facility.

The foregoing description of preferred embodiments of the invention has been presented for purposes of illustration and description. It is not intended to be exhaustive or to limit the invention to the precise form disclosed. Obvious modifications or variations are possible in light of the teachings in the art. The embodiments were chosen and described in order to best illustrate the principles of the invention and its practical application to thereby enable one of ordinary skill in the art to best utilize the invention in various embodiments and with various modifications as are suited to the particular use contemplated. One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A method for forming an endoluminal fold of tissue within a body cavity, comprising:
inserting an instrument having a flexible shaft and an end effector into a body cavity through a natural opening in a body, the end effector including a tissue receiving cavity and at least one electrode configured to deliver energy to tissue disposed in the tissue receiving cavity;
positioning the end effector adjacent tissue to be treated;
manipulating tissue to create a tissue fold that is disposed within the tissue receiving cavity; and
delivering energy to the tissue fold disposed within the tissue receiving cavity through the at least one electrode such that adjacent layers of the tissue that form the tissue fold are secured.

2. The method of claim 1, wherein the end effector includes first and second jaws that define the tissue receiving cavity.

3. The method of claim 1, wherein delivering energy comprises delivering RF energy.

4. The method of claim 1, wherein delivering energy comprises delivering ultrasonic energy.

5. The method of claim 2, wherein the at least one electrode comprises a needle movably disposed within one of the jaws, and wherein delivering energy comprises activating the needle after inserting the needle through at least a portion of the tissue that forms the tissue fold.

6. The method of claim 5, wherein delivering energy comprises contacting opposed serosal layers of the tissue fold and applying energy thereto effective to bond the opposed layers.

7. The method of claim 5, wherein the needle is inserted substantially along a central axis of the tissue fold.

8. The method of claim 5, wherein the needle is inserted substantially transverse to the tissue fold.

9. The method of claim 5, wherein the needle is inserted through both of the adjacent layers of the tissue that form the tissue fold.

10. The method of claim 1, wherein the end effector is movable between a delivery configuration and a treatment configuration, and wherein the tissue fold has a depth greater than a length of the tissue receiving cavity in the delivery configuration.

11. An endoscopic instrument, comprising:
an elongate, flexible shaft having proximal and distal ends;
an end effector disposed at the distal end of the shaft, the end effector having a tissue receiving cavity and at least one electrode associated therewith that is effective to deliver energy to tissue disposed in the tissue receiving cavity;
a tissue manipulator associated with the end effector, the tissue manipulator configured to engage tissue to enable tissue to be disposed within the tissue receiving cavity to create a tissue fold; and
an actuator effective to cause energy to be delivered through the at least one electrode to the tissue fold disposed within the tissue receiving cavity such that adjacent layers of the tissue that form the tissue fold can be secured.

12. The endoscopic instrument of claim 11, wherein the end effector comprises first and second jaws.

13. The endoscopic instrument of claim 11, wherein the electrode is configured to deliver RF energy.

14. The endoscopic instrument of claim 11, wherein the electrode is configured to deliver ultrasonic energy.

15. The endoscopic instrument of claim 14, wherein the electrode is movable from a first position substantially within one of the jaws, to a second position at least partially within the tissue receiving cavity.

16. The endoscopic instrument of claim 15, wherein the electrode is configured to penetrate tissue within the tissue receiving cavity as the electrode moves from the first position to the second position.

17. The endoscopic instrument of claim 11, wherein the electrode is configured to penetrate tissue substantially transverse to the tissue fold.

18. The endoscopic instrument of claim 11, wherein the electrode is configured to penetrate tissue substantially along a central axis of the tissue fold.

19. The endoscopic instrument of claim 11, wherein the electrode is configured to penetrate both of the adjacent layers of the tissue that form the tissue fold.

20. The endoscopic instrument of claim 11, wherein the end effector is movable from a delivery configuration to a treatment configuration, and wherein the tissue fold has a depth greater than a length of the tissue receiving cavity in the delivery configuration.
